(19) United States
(12) Patent Application Publication
(10) Pub. No.: US 2016/0074533 A1
(43) Pub. Date: Mar. 17, 2016

(54) VECTOR ENCODING THERAPEUTIC POLYPEPTIDE AND SAFETY ELEMENTS TO CLEAR TRANSDUCED CELLS

(71) Applicant: University Health Network, Toronto (CA)

(72) Inventor: Jeffrey A. Medin, North York (CA)

(21) Appl. No.: 14/949,639

(22) Filed: Nov. 23, 2015

Related U.S. Application Data
(63) Continuation of application No. 12/532,572, filed on Sep. 22, 2009, now abandoned.

(30) Foreign Application Priority Data
Mar. 27, 2007 (CA) 2584494

(51) Int. Cl. A61K 48/00; C12N 15/86

(52) U.S. Cl. CPC A61K 48/005; C12N 15/86; C12N 2750/14143; C12N 2740/17043; C12N 2740/15043; C12N 2750/14133; C12N 2740/17033; C12N 2740/15033

(57) ABSTRACT

A composition comprising: a stably integrating delivery vector; a modified mammalian thymidylate kinase (tmpk) activator polynucleotide wherein the modified mammalian tmpk polynucleotide encodes a modified mammalian tmpk polypeptide that increases phosphorylation of a prodrug relative to phosphorylation of the prodrug by wild-type mammalian tmpk polypeptide to a drug; and/or a targeting polynucleotide encoding a cell surface polypeptide that selectively binds a toxic binding agent. The disclosure also relates to use of these compositions in methods of treatment of diseases such as Fabry disease.

### RELATED APPLICATIONS

This application is a continuation of U.S. application Ser. No. 12/532,572, filed on Sep. 22, 2009, which is a National stage entry of International Application No. PCT/CA2008/000579 filed on Mar. 27, 2008, which claims the benefit of Canadian Application serial no. 2,584,494, filed on Mar. 27, 2007, each of these applications being incorporated herein by reference in their entirety.

### GOVERNMENT INTEREST

These studies were supported in part by a grant from the National Institutes of Health (HL70569). The United States government may have rights in this disclosure.

### INCORPORATION OF SEQUENCE LISTING

A computer readable form of the Sequence Listing “10723-P5613US01_SL.txt” (81,920 bytes), submitted via EFS-WEB and created on Nov. 23, 2015, is herein incorporated by reference.

### FIELD OF THE APPLICATION

The disclosure relates to compositions comprising a vector encoding safety elements to clear transduced cells.

### BACKGROUND OF THE APPLICATION

Gene therapy has been used successfully to treat a number of inherited disorders.1,2 Although many viral and non-viral gene delivery alternatives exist, retroviral vectors offer the advantages of stable integration into host genomes, the ability to infect a wide variety of cell types, and relatively high levels of transgene expression.3 Concerns regarding the safety of integrating vectors have been prompted, however, by the development of leukemia in three X-linked severe combined immunodeficiency patients in a recent clinical trial using an oncoretroviral vector.4 A variety of explanations for this outcome have been proposed, but the exact mechanism of leukemogenesis has remained unresolved, as no other clinical trials have reported this type of adverse event.5,6

Despite this outcome, retroviral gene therapy continues because of the conceptual effectiveness of the treatment and the fact that gene therapy is the only potential cure available for many disorders such as X-linked severe combined immunodeficiency.

Integrating viral vectors are still a good choice for gene therapy because they offer fairly efficient transduction and consistent long-term gene expression. Much research has been directed towards improving vector design to increase safety and reliability. Therefore, the development of improved vectors and viable alternative safety strategies is exceedingly important and timely.

One example of a disease targeted for gene therapy is Fabry disease, a lysosomal storage disorder resulting from a deficiency of α-galactosidase A (α-gal A) activity. Fabry disease is a good candidate for gene therapy because there is reduced neurological involvement in contrast to many other lysosomal storage disorders, and supra-physiological levels of α-galA are well-tolerated.8

Gene therapy for Fabry disease by introducing aα-galactosidase A (α-gal A) activity, has the potential to provide a cure for the disorder with a single treatment. Despite modifications to existing vectors, concerns have arisen regarding the risk of genotoxicity associated with the use of retroviruses. There remains a need for suitable gene therapy vectors for Fabry disease and other enzyme deficiency diseases.

### SUMMARY OF THE INVENTION

Incorporating an effective suicide gene into a therapeutic vector ensures that any malignant clones arising from deleterious insertion of the vector are specifically killed. Likewise, such a control schema is useful as an inserted safety component for a variety of transplants, including stem cell transplants reducing teratomas, for example, should these outgrowth events develop. A suicide gene schema us also useful to control post-transplant complications such as Graft v Host disease. The invention provides vectors with improved safety elements to effectively clear transduced cells to further decrease risk to the patient.

The disclosure provides a novel strategy for improving the safety of therapeutic integration vectors. This novel strategy has great utility, as a variety of cell surface proteins are readily incorporated into various retroviral vectors in combination with any therapeutic transgene. Using this system adds another safety mechanism to current and future retroviral gene transfer systems and transplant schemas of a variety of manifestations.

The disclosure provides a composition comprising:

a stably integrating delivery vector;

an activator polynucleotide encoding a polypeptide that converts a prodrug to a drug; and/or

a docking polynucleotide encoding a docking polypeptide that selectively binds a toxic binding agent.

In one embodiment, the activator polynucleotide is deoxycytidine kinase. In one embodiment the activator polynucleotide is thymidylate kinase. In another embodiment the activator polynucleotide is a modified thymidylate kinase. In another embodiment the activator polynucleotide is thymidine kinase (tk). In a further embodiment the tk is herpes simplex virus-tk (HSV-tk). In another embodiment, the tk is Equine Herpes Virus Type 4 (EHV4-tk). Mutations, variants, and derivatives thereof that maintain kinase activity are also included.

The application further provides a suicide gene therapy safety system comprising a stably integrating delivery vector comprising an activator polynucleotide encoding a polypeptide that converts a prodrug to a drug and/or a docking polynucleotide encoding a docking polypeptide that selectively binds a toxic binding agent. In one embodiment the docking polypeptide is a cell surface protein. The system further comprises, a prodrug that is converted to a drug by the activator polynucleotide and a toxic binding polypeptide that binds the docking polypeptide. In one embodiment, the docking polynucleotide is a polynucleotide that encodes a cell surface polypeptide or cell surface marker. In one embodiment, the docking polynucleotide is CD25. In another embodiment, the docking polynucleotide is truncated CD19. In other embodiments, the docking polynucleotide is selected from the group consisting of CD19, truncated CD19, EGFP, CD25, LNGFR, truncated LNGFR, CD24, truncated CD34, EpoR, HSA and CD20.

In one embodiment the toxic binding agent is an antibody. In another embodiment, the toxic binding agent is an antibody conjugated to a toxin. In certain embodiments the toxin comprises saporin, other cytotoxic polypeptides, cytotoxic chemicals, radionuclides, etc. In a further embodiment, the antibody is an anti-CD25 antibody. In another embodiment, the antibody is an anti-CD19 antibody. In other embodiments, the antibody binds CD19, truncated CD19, EGFP, CD25, LNGFR, truncated LNGFR, CD24, truncated CD34, EpoR, HSA or CD20.

In one embodiment, the delivery vector is an integrating vector. In another embodiment, the delivery vector is a retroviral vector such as an oncoretroviral or lentiviral vector. In other embodiments, the delivery vector is a foamy virus. In yet other embodiments, the delivery vector is a transposon such as Sleeping Beauty (Discovery Genomics, Inc.; U.S. Pat. No. 6,489,458).

The disclosure also provides a method of expressing an activator polynucleotide and a docking polynucleotide in a mammalian cell comprising contacting the mammalian cell with a composition of the disclosure. In other embodiments, the disclosure provides a method of additionally expressing a therapeutic polypeptide.

In one embodiment the mammalian cell is selected from the group comprising a stem cell, a hematopoietic cell, a T cell and a human cell.

Any stem cell or ES cell or iPS cell that is transplanted benefits from having this safety system to decrease the risk of aberrant cell growth when cells are placed out of their normal context. A therapeutic gene is optionally provided. The safety system described herein is also useful in BMT and DLI. One can use direct tumor injection to administer the safety system herein described; addition of the prodrug will then kill transduced and neighboring cells.

The system is also useful to remove any transplanted cell in any transplantation setting that has lost effectiveness or actually becomes deleterious to the host by any mechanism.

In another embodiment, the application discloses compositions and systems further comprising a therapeutic polynucleotide. In one embodiment the therapeutic polynucleotide is α-galactosidase A(αGal A).

Another aspect of the disclosure provides a kit comprising the composition or system previously described, optionally additionally comprising instructions for use according to a method described herein.

Other features and advantages of the present disclosure will become apparent from the following detailed description. It should be understood, however, that the detailed description and the specific examples while indicating preferred embodiments of the disclosure are given by way of illustration only, since various changes and modifications within the scope of the disclosure will become apparent to those skilled in the art from this detailed description.

### BRIEF DESCRIPTION OF THE DRAWINGS

Preferred embodiments of the disclosure will be described in relation to the drawings in which:

FIG. 1 In vitro clearance of C1498 cells expressing a broad concentration range of human CD25 (huCD25) molecules by anti-Tac saporin (ATS). C1498 cells were infected with LV/α-gal A/huCD25 and then sorted by magnetic activated cell sorting to isolate a pool of cells that express huCD25. Shown are two cell populations that are (a) 90% and (b) 45% positive for huCD25 expression as measured by flow cytometry analysis. Cells were treated with 5 nM of each reagent. (c, d) Cell proliferation was assessed by MTT [3-(4,5-dimethyl-2-thiazolyl)-2,5-diphenyl-2H-tetrazolium bromide] assays 72 hours later. (e, f) Cytoxicity was assessed by measurement of lactate dehydrogenase (LDH) release 48 hours later. AT, anti-Tac; SAP, saporin; IgG-SAP, IgG-saporin (isotypec control immunotoxin). Error bars represent SD. *P<0.05, **P<0.01, ***P<0.001 for ATS compared with all other groups.

FIG. 2 In vitro clearance of a C1498/CD25 clone by anti-Tac-saporin (ATS). (a) Representative flow cytometry analysis of a derived single cell clone of C1498/huCD25 cells. Cells were transduced with LV/α-gal A/CD25 and single-cell clones were isolated by flow cytometry on the basis of human CD25 (huCD25) expression. (b) Proliferation of C1498/huCD25 and non-transduced (NT) cells after incubation with ATS or control reagents for 72 hours, as measured by MTT [3-(4,5-dimethyl-2-thiazolyl)-2,5-diphenyl-2H-tetrazolium bromide] assay. (c) Cell death, measured by a lactate dehydrogenase (LDH) release assay. Error bars represent SD. ***P<0.001 for ATS compared with all other groups.

FIG. 3 The in vivo effect of different antibody doses on plasma human CD25 (huCD25) levels. Fabry mice were transplanted with 1×10⁶ C1498/huCD25 cells and treated with 5 μg anti-Tac-saporin (ATS), 20 μg ATS, or 20 μg saporin (SAP) 2 days after cell transplantation. Plasma was collected from the peripheral blood 18 days after cell transplantation and analyzed for levels of soluble huCD25. n=3 per group.

FIG. 4 Anti-Tac-saporin (ATS) and anti-Tac (AT) treatment in a human CD25 (huCD25)-expressing myeloid leukemia model. Fabry mice were transplanted with C1498/huCD25 cells and treated with immunotoxins on days 2, 4, and 6. On day 18, plasma was analyzed for (a) soluble huCD25 levels by enzyme-linked immunosorbent assay and (b) α-galactosidase A (α-gal A) activity. Error bars represent SEM. n=6 in all groups, except for the untreated group (n=8) and the wildtype (WT) group (n=4). (c) Kaplan-Meier survival curve for treated and control mice.

FIG. 5 Bone marrow transplantation model. Bone marrow mononuclear cells (BMMNCs) were harvested from Fabry mice and transduced using supernatant from E86/pMFG/α-gal A/IRES/huCD25 clone 21 (n=24) or E86/pUMFG/enYFP (n=6). Forty-eight hours after transduction, BMMNCs were analyzed for expression of (a) human CD25 (huCD25) or (b) enhanced yellow fluorescent protein (enYFP). Transduced cells were transplanted into lethally irradiated recipient Fabry mice. (c) Eight weeks after transplant, plasma of recipient mice was analyzed for α-galactosidase A (α-gal A) activity. Error bars represent SEM.

FIG. 6 Clearance of retrovirally transduced bone marrow-derived cells by anti-Tac-saporin (ATS) and anti-Tac (AT). Nine weeks after bone marrow transplantation with cells transduced with either E86/pMFG/α-gal A/IRES/huCD25 clone21 or E86/pUMFG/enYFP, mice were treated with either ATS, AT, or immunogloblin (Ig)G Ab conjugated to SAP (IgG-SAP). Peripheral blood was collected 1 week later and analyzed for (a) levels of soluble human CD25 (huCD25) in the plasma and (b) expression of huCD25 on mononuclear cells. Values are expressed as percentage reduction compared with pre-treatment values (measured at week 8). (c) Expression of enhanced yellow fluorescent protein (enYFP) on peripheral blood mononuclear cells (PBMNCs) over the course of the experiment. Error bars represent SEM. n=5 in all groups except ATS (n=4) and green fluorescent protein (n=6).

FIG. 7 Systemic effect of anti-Tac-saporin (ATS) treatment on α-galactosidase A (α-gal A) activity. Twelve weeks after bone marrow transplantation and three weeks after the first treatment with immunotoxin, mice were killed and α-gal activity was measured in various tissues: (a) peripheral blood mononuclear cells, (b) liver, (c) spleen. Error bars represent SEM. n=5 in all groups except ATS (n=4) and green fluorescent protein (n=6).

### DETAILED DESCRIPTION OF THE INVENTION

The disclosure relates to the use of a cell surface antigen such as huCD25 in a gene expression cassette as a safety mechanism for retroviral vectors.

The inventors have demonstrated that a targeted antibody and/or targeted immunotoxin reduces tumor burden and selectively clear transduced hematopoietic cells that express a target antigen, thus acting as a built-in safety mechanism for gene therapy vectors. The inventors show that anti-CD25 antibody and/or an anti-CD25 conjugated immunotoxin, specifically targets and eliminate transduced leukemia cells expressing CD25.

In one embodiment, the disclosure provides a combination of a novel prodrug/enzyme and a docking polypeptide/toxic binding agent for suicide gene therapy, for example for use in transplant schemas. The disclosure also provides the combination of a therapeutic gene, a novel prodrug/enzyme and a docking polypeptide/toxic binding agent for gene therapy. In one embodiment, catalytically improved variants of human tmpk and a CD25 docking polynucleotide are delivered into target cells by novel lentiviruses (LVs) providing the ability to selectively clear these cells in vitro and in vivo by administering the prodrug AZT and/or a CD25 toxic binding agent. Catalytically improved variants of human tmpk, methods of delivering said modified variants are disclosed in U.S. Ser. No. 11/559,757, U.S. Ser. No. 12/052,565 filed Mar. 20, 2008 and U.S. provisional application 61/038,398 filed Mar. 20, 2008 each of which are herein incorporated by reference in its entirety and in Sato et al. Engineered Human tmpk/AZT As a Novel Enzyme/Prodrug Axis for Suicide Gene Therapy. Mol Ther. 2007 doi:10.1038/mt.sj.6300122. Other genes, such as dck, HSV-tk, EHV4-tk and derivatives, are useful with other prodrugs. In addition, a cell surface protein (marker), such as truncated CD19, CD19, CD20, HSA, truncated LNGFR, CD34, CD24 or CD25—is delivered into target cells which allows for detecting and/or isolating transduced cells and can further provide the ability to selectively clear these cells in vitro and in vivo by administering a toxic binding agent such as an antibody alone or comprised in an immunotoxin (antibody conjugated to a toxin) directed against the docking polypeptide such as a cell surface protein.

In an alternate embodiment the activator polynucleotide and docking polynucleotide are fused so as to produce a fusion polypeptide upon expression. In one embodiment, truncated CD19 is fused to a modified mammalian tmpk. As the docking polynucleotide is fused to the activator polynucleotide such as mammalian modified tmpk, permissive cells transfected or transduced with such a construct will express tmpk and the docking polynucleotide. This is useful for a number of applications including ensuring that all cells isolated using the docking polynucleotide express both the tmpk safety component and the docking polypeptide safety component. A docking polynucleotide fused to tmpk is alternatively referred to as tmpk/docking polynucleotide fusions.

These suicide genes are efficiently transferred into mammalian T cells and cell lines. In other embodiments, these suicide genes are efficiently transferred into ES cells IPS cells, mesenchymal stem cells, bone marrow stroma cells, endothelial progenitor cells, hematopoietic stem cells, any other stem cell for transplantation, etc.

The disclosure provides the first gene therapy methods and vectors using both suicide genes and docking polypeptides such as encoded by cell surface genes recognized by immunotoxins to provide more effective clearance of transduced cells. In addition, this system is useful to endow stem cells (both embryonic and of later ontogeny) for in clinical transplantation, for example, with a reliable safety system.

### Safety Systems and Vector Constructs

The disclosure provides safety systems comprising combinations of a novel prodrug/enzyme and a docking polypeptide/toxic binding agent for suicide gene therapy. Such safety systems are useful, for example in clearing cells in transplant schemas, for example in the event of a transplant adverse event. The disclosure also provides the combination of a therapeutic gene, a novel prodrug/enzyme and a docking polypeptide/toxic binding agent for gene therapy. Certain embodiments of the disclosure optionally comprise a vector construct including i) an activator gene and/or ii) DNA encoding a cell surface protein recognized by a toxic binding agent.

### i) The Activator Gene—Conversion of Prodrug to Drug to Kill Transduced Cells

The term “activator gene” or “activator polynucleotide” also referred to as a ‘cell fate control gene’ as used herein refers to a safety element comprising a polynucleotide encoding a polypeptide that catalytically converts or aids in the conversion of a prodrug to a drug, such that administration of the prodrug is cytotoxic to cells expressing the activator gene. The term “suicide gene” is used interchangeably with “activator gene” herein.

The activator genes of the disclosure such as modified mammalian tmpk work by increasing phosphorylation of prodrugs such as AZT. For example, the prodrug AZT is converted through a series of phosphorylation steps into AZT-triphosphate (AZT-TP)¹². This is the active metabolite that inhibits replication of the human immunodeficiency virus (HIV)¹³⁻¹⁵, and to a lesser extent, DNA replication in eukaryotic cells¹⁶. Safety profiles for this compound are well known and concentrations of AZT in the bloodstream of AIDS patients being treated with this agent can reach high levels. The rate-limiting step in the conversion of AZT to the toxic AZT-TP form is the intermediate step of phosphorylation of AZT-monophosphate (AZT-MP) to AZT-diphosphate (AZT-DP) catalyzed by the cellular thymidylate kinase (tmpk), which has a low enzymatic efficiency for AZT-MP¹⁷. Accumulation of AZT-metabolites in the cells of AZT-treated AIDS patients reportedly induces toxic mitochondrial myopathy¹⁸⁻²². To harness this dual toxicity of AZT-TP, the disclosure uses any suitable suicide gene encoding a polypeptide that converts prodrug to drug.

### Tmpk

An example of a useful safety element comprises a nucleic acid encoding mammalian or human tmpk. In order to improve the processing of AZT-MP to AZT-DP, thereby increasing intracellular AZT-TP concentrations, minimally modified tmpk mutants with approximately 200-fold enhanced activity for AZT-MP have been engineered (Brundiers R, Lavie A, Veit T, Reinstein J, Schlichting I, Ostermann N, et al. Modifying human thymidylate kinase to potentiate azidothymidine activation. J Biol Chem. 1999; 274: 35289-35292; Ostermann N, Lavie A, Padiyar S, Brundiers R, Veit T, Reinstein J, et al. Potentiating AZT activation: structures of wild-type and mutant human thymidylate kinase suggest reasons for the mutants' improved kinetics with the HIV prodrug metabolite AZTMP. J Mol Biol. 2000; 304: 43-53).

Thymidylate kinase is a kinase that catalyzes the addition of a phosphoryl group to thymidylate as well as thymidine analogs such as AZT. Several wild-type human sequences have been reported. SEQ ID NOS: 1, 3, 5 and 7 are reported nucleotide sequences of human thymidylate kinase (SEQ ID NO: 7 does not have a stop codon). The different sequences represent natural polymorphic variations present in the population and it will be recognized in the art that future identified molecules with polymorphic variations will also be considered to be wildtype tmpk. SEQ ID NO: 9 is the reported mouse thymidylate kinase sequence. The mouse sequence shares 82% nucleotide identity, 81% amino acid identity and several residues that have been identified as limiting the nucleoside analog activity of the human tmpk enzyme and which result in increased enzymatic activity when modified, are conserved in the murine sequence. The corresponding amino acid sequences are reported in SEQ ID NOS: 2, 4, 6, 8, and 10. SEQ ID NO: 2 provides the amino acid sequence for the wild-type tmpk polynucleotide described in SEQ ID NO: 1; SEQ ID NO: 4 provides the amino acid sequence for the wild-type tmpk polynucleotide reported in SEQ ID NO: 3, SEQ ID NO: 6 provides the amino acid sequence for the wild-type tmpk polynucleotide described in SEQ ID NO: 5; SEQ ID NO: 8 provides the putative sequence of the wild-type tmpk polynucleotide reported in SEQ ID NO: 7; and SEQ ID NO: 10 provides the amino acid sequence of the wild-type murine tmpk polynucleotide described in SEQ ID NO: 9. Modified tmpk molecules and mutant tmpk refer to mammalian tmpk molecules that have been modified compared to wild-type. Among the mutant tmpks, some of these showed a superior enzymatic activity to convert deoxy-thymidine-monophosphate (dTMP) to dTMP-diphosphate (dTDP) or AZT-MP to AZT-DP. Increased kinase activity relative to wild-type refers to modified tmpk molecules that exhibit improved enzymatic kinetics compared to tmpk wild-type. The improved activity comprises increases in binding and or enzymatic turnover to convert the monophosphate-form of the substrate of tmpk to the diphosphate form.

Mutations which show superior enzymatic activity included the F105Y mutant (SEQ ID NO: 11, SEQ ID NO: 21), R16GLL mutant (SEQ ID NO: 12, SEQ ID NO: 22) and the R200A mutant (SEQ ID NOS: 15 and 16).

One aspect of the invention provides delivery vectors comprising modified tmpk enzymes with increased nucleoside analog kinase activity relative to wild-type. In one aspect, the modification that increases tmpk nucleoside analog kinase activity comprises one or more deletions. The deletions are optionally internal or optionally result in a truncated variant. In an alternate embodiment the modification that increases tmpk nucleoside analog kinase activity comprises one or more point mutations. In another embodiment an exogenous sequence replaces an endogenous sequence. For example, in one embodiment all or part of the large lid domain of human tmpk (SEQ ID NO:20) is replaced with all or part of the large lid domain of a different species. In one embodiment the different species is a bacteria species. In one embodiment, all or part of the large lid domain of human tmpk (SEQ ID NO:20) is replaced with all or part of the large lid domain of E. coli tmpk (SEQ ID NO:17). In another embodiment, residues 145-148 of SEQ ID NO: 1 (AFGH) are replaced with all or part of the small lid region of E. coli residues 151-156 in SEQ ID NO: 17 (RARGEL). In another embodiment the modified tmpk is selected from the group including the F105Y mutant (SEQ ID NO: 11, SEQ ID NO: 21), R16GLL mutant (SEQ ID NO: 12, SEQ ID NO: 22), a tmpk molecule modified by the substitution of all or part of a bacterial large lid domain such as the E. coli large lid domain in SEQ ID NO: 17, a tmpk molecule modified by the substitution of all or part of a bacterial small lid domain such as the E. coli small lid domain at 151-156 of SEQ ID NO: 17, and the R200A mutant (SEQ ID NOS: 15 and 16).

In another embodiment, the exogenous sequence is optionally synthesized or obtained from a non-mammalian thymidylate kinase such as a bacterial thymidylate kinase. As used herein a modified mammalian tmpk molecule includes a modified tmpk molecule that comprises non-mammalian sequences such as all or part of either a large lid domain or a small lid domain sequence from bacteria such as E. coli. A variant may comprise one or more of the aforementioned modifications. Examples of modifications are described above.

A person skilled in the art will recognize that conservative amino acid substitutions as well as additions/deletions or a number of divergent amino acid sequences can be used are readily made to the disclosed sequences and are within the scope of the present disclosure.

A “conservative amino acid substitution” as used herein, is one in which one amino acid residue is replaced with another amino acid residue without abolishing the protein's desired properties. Conservative amino acid substitutions are known in the art. For example, conservative substitutions include substituting an amino acid in one of the following groups for another amino acid in the same group: alanine (A), serine (S), and threonine (T); aspartic acid (D) and glutamic acid (E); asparagine (N) and glutamine (Q); arginine (R) and lysine (L); isoleucine (I), leucine (L), methionine (M), valine (V); and phenylalanine (F), tyrosine (Y), and tryptophan (W).

Also included are tmpk sequences with sequence identity with the tmpk sequences provided below. In one embodiment, the tmpk has 60-70%, 70-80%, 90-95%, 95-99% or 99-99.9% sequence identity with a tmpk described herein.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

Phosphorylation of the prodrug leads to its activation and increases its effectiveness in killing vector transduced cells (also called “suicide gene therapy”). The disclosure is useful in the event of a transplant related adverse event. A transplant related adverse event typically comprises graft versus host disease where following T-cell (or other cell) transplant to a recipient the transplanted cells attack the host. A transplant adverse event also comprises any situation where it would be beneficial to eliminate the transplanted cells, including where transplanted cells contain integrations that can cause malignant transformation or any other disease. The transplanted cells express mutant tmpk so that upon detection of graft versus host disease, a prodrug such as AZT is optionally administered to the patient to kill the transplanted cells.

### Other Activator Molecules

Other genes are useful with other prodrugs. Nucleic acid encoding dck is one example of a useful gene. The dck polypeptide catalyzes the phosphorylation of a range of pyrimidine and purine deoxynucleotides to the corresponding nucleotide to modify prodrug compounds so that they exhibit an antineoplastic effect, such as ara-C, aza-CdK, dFdC, cladribine, zalcitabine and fludarabine (see eg. U.S. Pat. No. 6,423,692). In addition, herpes simplex virus type 1 thymidine kinase (HSV-tk), Equine Herpes Virus 4 thymidine kinase (EHV4-tk) and their derivatives are also useful as suicide genes. The tk gene, converts the antiviral prodrug ganciclovir (GCV) to its toxic drug form. It converts GCV to GCV-MP; this is converted by guanylate kinase to GCV-DP. This is converted by other cellular kinases to GCV-TP, which intercalates into DNA upon upon replication causing termination and eventual cell death. As mentioned above DCK converts a variety of drugs to a mono-phosphorylated form.

ii) The Docking Polypeptide—Use of a Toxic binding agent to Kill Transduced Cells

The term “docking polynucleotide” or “docking gene” alternatively referred to as “targeting polynucleotide” or “targeting gene” as used herein refers to a polynucleotide that encodes a polypeptide (herein referred to as a docking polypeptide) that functions as a cell marker and is accessible to binding by a toxic binding agent such as an antibody or an immunotoxin. The docking polynucleotide can comprise a polypeptide that protects cells from a different drug—such as neomycin phosphotransferase and G418. In certain embodiments, the docking polynucleotide encodes a cell surface polypeptide. The polynucleotide optionally provides for a mode of isolating cells expressing said docking molecule. The docking molecule is optionally used to select transduced or transfected cells or to determine the efficiency of cell transduction or transfection.

A good docking gene component optionally encodes a polypeptide that is recognized by an antibody and is useful for enrichment, sorting, tracking, and also killing such as a cell surface molecule. Such docking gene components optionally have the additional ability to track cells and ensure that expression of the therapeutic safety gene is maintained. A variety of cell surface markers are useful in this context: human CD24, murine HSA, human CD25 (huCD25), a truncated form of LNGFR, and truncated CD34.

As the docking polypeptide is substantially overexpressed in transduced cells, said transduced cells are targeted due to mass action effects, i.e. more conjugated toxin will accumulate on the cells that express more of the cell surface marker.

CD19 (SEQ ID NOS: 27-28) is a 95-kDa glycoprotein of the immunoglobulin superfamily. It forms a complex with CD21, CD81, and Leu-13, and collectively functions to modulate the activation threshold of the B cell receptor. As expression of CD19 and CD21 is restricted to B cell lineages from immature progenitors to blasts, it is suitable for use in murine and human T cells. To further decrease any signaling capacity from the CD19 molecule, the cytoplasmic tail has been deleted for the present adaptation. In one embodiment truncated CD19 comprises all or a portion of SEQ ID NO: 29. In another embodiment truncated CD19 comprises all or a portion of SEQ ID NO: 30. In another embodiment truncated CD19 comprises all or a portion of SEQ ID NO: 31.

Molecules that are useful as cell markers or detection agents comprise CD19, truncated CD19, CD25 and EGFP, HSA, CD20, GFP, ETC. EGFP is variably referred to as enGFP or GFP herein. One skilled in the art will recognize that other fluorescent molecules are similarly used. These molecules are optionally fused to tmpk to provide a tmpk/docking fusion molecule.

As mentioned, the docking polynucleotide encodes a molecule that can be used to isolate transduced or transfected cells. The docking polynucleotide useful in vectors optionally comprises modified tmpk or control molecules. Control molecules include molecules that do not function as suicide gene therapy molecules which are typically employed to assess the effect of tmpk mutants in similarly related cells.

In certain embodiments of the disclosure, the docking polynucleotide encodes a cell surface protein (marker), such as truncated CD19, CD19, CD20 or CD25 is delivered into target cells which provides the ability to selectively clear these cells in vitro and in vivo by administering a toxic binding agent such as an antibody or fragment thereof or an immunotoxin directed against the cell surface protein. The toxic binding agent binds and kills transfected or transduced cells expressing the docking polynucleotide.

The phrase “cell surface protein” or “cell surface polypeptide” as used herein refers to a polypeptide that is expressed, in whole or in part on the surface of a cell. This optionally includes polypeptide fragments that are presented on cells as well as polypeptides or fragments thereof that are naturally found on the surface of a cell. In the context of a cell modified to express a vector construct comprising a docking polypeptide, wherein the docking polypeptide is a cell surface polypeptide, the cell surface marker need not be native to the cell it is being expressed on.

The term “kills” with respect to transfected or transduced cells refers to inducing cell death through any of a variety of mechanisms including apoptosis, necrosis and autophagy. For example an agent that is cytotoxic kills the cells.

The term “toxic binding agent” as used herein refers to an agent that binds a docking polypeptide expressed on or in a cell transfected or transduced with a composition, system or vector construct described herein, and which is cytotoxic to and/or kills said cell.

The inventors have shown that cell clearance is attainable using the AT antibody and/or the ATS immunotoxin.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. The term also includes antibodies or antibody fragments that bind to the docking polypeptides disclosed herein. A number of clinical antibodies are known in the art that can be used with the methods of the application. For example Herceptin which recognizes a cell surface molecule called HER2/neu can be employed. Other drugs exist here that recognize CD25 and CD19: Br J Haematol. 2006 July; 134(2):157-70. Epub 2006 Jun. 12 The anti-CD20 antibody rituximab augments the immunospecific therapeutic effectiveness of an anti-CD19 immunotoxin directed against human B-cell lymphoma. Flavell D J, Warnes S L, Bryson C J, Field S A, Noss A L, Packham G, Flavell S U; Clin Cancer Res. 2005 May 1; 11(9):3567-73 Anti-CD19-targeted liposomal doxorubicin improves the therapeutic efficacy in murine B-cell lymphoma and ameliorates the toxicity of liposomes with varying drug release rates, Allen T M, Mumbengegwi D R, Charrois G J.; Neurodegener Dis. 2008; 5(1):23-6, Humanized anti-CD25 antibody treatment with daclizumab in multiple sclerosis. Martin R.

Monoclonal antibodies against a variety of receptors and molecules are currently being introduced in clinical medicine. One of these targets is the interleukin-2 receptor alpha-chain CD25. The humanized monoclonal anti-CD25 antibody daclizumab (Zenapax) has been approved several years ago for the prevention of allotransplant rejection and adult T cell leukemia. Following promising observations in uveitis, daclizumab has been tested in a number of small clinical trials in multiple sclerosis based on the rationale that blocking CD25 would prevent the expansion of autoreactive T lymphocytes. The data from this preliminary clinical exploration as well as findings about the mechanism of action of anti-CD25 treatment are summarized in this study. Copyright (c) 2008 S. Karger A G, Basel.

The term “immunotoxin” as used herein means an antibody or fragment thereof that is cytotoxic and/or an antibody or fragment there of that is fused to a toxic agent. Immunotoxins are described in this application and known in the art, for example, in US patent application publication no. 20070059275.

Many immunotoxins are approved for use in humans. In one embodiment the immunotoxin is a murine anti-Tac (AT) monoclonal antibody19 fused to saporin (SAP),20 a toxin that irreversibly damages ribosomes by cleaving adenine molecules from ribosomal RNA.21 The inventors have demonstrated both in vitro and in vivo that AT and the AT-SAP (ATS) complex specifically targets and kill retrovirally transduced cells that express huCD25. Importantly, the inventors have achieved enzymatic correction of a mouse model of Fabry disease using a bicistronic vector of the disclosure and demonstrate removal of transduced cells using both ATS and AT. As noted above, the disclosure provides in one embodiment the combination of a therapeutic gene, a novel prodrug/enzyme and a docking gene/toxic binding agent for transducing cells and/or clearing transduced cells, for example in gene therapy. Catalytically improved activator polynucleotide, such as dck or variants of human tmpk, were delivered into target cells by novel lentiviruses (LVs), and the ability to selectively clear these cells in vitro and in vivo in response to increasing AZT concentrations was thoroughly evaluated. The inventors transfer these suicide genes and cell surface protein into cells, such as mammalian T cells and cell lines, preferably human T-cells and cell lines.

Accordingly, the disclosure relates to methods of using a suicide gene therapy system comprising an activator polynucleotide and/or docking polynucleotide inserted in transplant cells for treatment of diseases such as Fabry disease, Farber disease, cancer and controlling transplant-associated graft versus host disease. Where the disease being treated results from a gene or enzyme deficiency, the system optionally comprises a therapeutic gene for gene therapy. For example, the system used for the treatment of Fabry disease further comprises a therapeutic gene, such as alpha-galactosidase A. The system used for the treatment of Farber disease further comprises a therapeutic gene, acid ceramidase. A lentivirus is optionally used to deliver an activator polynucleotide and docking polynucleotide. Other methods of delivery are also useful for example onco-retroviral vectors that engineer expression of huCD25 (see Qin et al. and Medin PNAS 2004).

The compositions and systems disclosed are useful in the event of a transplant related adverse event. A transplant related adverse event optionally comprises a graft versus host disease where following T-cell (or other cell) transplant to a recipient the transplanted cells attack the host. A transplant adverse event also comprises any situation where it would be beneficial to eliminate the transplanted cells, including where transplanted cells comprise integrations that can cause disease. In the case of a transplant adverse event, the transplanted cells modified to comprise an activator polynucleotide, are treated with a prodrug that is converted to a cytotoxic drug by the enzyme encoded by the activator polynucleotide thereby resulting in cell death. In another embodiment, transplanted cells modified to comprise targeted polynucleotide are treated with an amino toxin that binds a polypeptide encoded by the docking polynucleotide thereby resulting in cell death. In a further embodiment, transplanted cells which are modified to comprise both an activator polynucleotide and a docking polynucleotide are treated with both a prodrug and an immunotoxin, such that a dual suicide safety system is utilized. The methods, compositions and systems of the disclosure are also useful to terminate transplanted cells once their primary desired functions are depleted: such as for facilitating transplantation of allogeneic organs, facilitating engraftment of hematopoietic stem cells, or directly attacking solid tumors or leukemias.

In one embodiment, a prodrug such as AZT is administered to the patient to kill the transplanted cells. In another embodiment, a toxic binding agent, such as an antibody or an antibody conjugated to a toxin, is administered to the subject and bind to the polypeptide, such as a cell surface polypeptide (eg. CD19, CD20, CD25), produced by a docking polynucleotide. The toxic binding agent is optionally administered before, concurrently with, or after administration of the prodrug.

For cancer treatment, the above method is useful to treat leukemia where donor transplant cells are used to kill leukemic cells. The transplanted cells expressing activator polynucleotide and docking polynucleotide are likely to also attack the host, so the disclosure allows the transplanted cells to be killed after detection of the onset of graft versus host disease.

In a variation of the disclosure, vectors comprising activator polynucleotide and docking polynucleotide are inserted directly into the solid tumor. Expression of activator polynucleotide to produce activator polypeptide sensitizes the immediate cells, and also surrounding cells if a ‘bystander effect’ is present as it is with some enzyme/prodrug combinations such as HSV-tk/GCV, etc., to the prodrug and expression of docking polynucleotide sensitize the cells to toxic binding agent.

Additionally, the activator polynucleotides and docking polynucleotide are useful as a general ‘safety component’ in gene therapy. For example in patients with Severe Combined Immunodeficiency Disease (SCID), gene therapy has been used successfully to introduce deficient genes however at least one clinical trial was halted due to safety concerns arising from inappropriate DNA integrations. The prior art also includes much discussion about the dangers of gene therapy due to vector integrations that can cause cancer. The safety component overcomes this problem by allowing the transplanted cells to be destroyed upon administration of a prodrug or a toxic binding agent.

(iii) Activator/Docking Fusions

In an alternate embodiment, the activator polynucleotide is fused to the docking polynucleotide to produce a fusion polypeptide upon expression. The inventors have made an activator/docking fusion by fusing truncated CD19 with a modified mammalian tmpk polynucleotide as described in U.S. provisional 61/038,398 filed Mar. 20, 2008 herein incorporated by reference. As the fusion is expressed in all cells, all cells express the docking polypeptide and the suicide gene product, the fusion construct provides a dual safety mechanism whereby each of the modified cells is killed by either a prodrug alone, a toxic binding agent alone or a combination thereof. This provides flexibility and ensures that all cells modified to express the fusion are killed if required.

The activator/docking fusion is optionally constructed comprising a tmpk activator. The tmpk component is optionally a N-terminal (or 5′) or C-terminal (or 3′) in continuous or discontinuous relationship to the docking component. For example, in a continuous relationship the fusion polypeptide can comprise a tmpk component fused to a docking polypeptide (e.g. NH2-tmpk-GFP-COOH) or alternatively can comprise a docking polypeptide component fused to a tmpk molecule (e.g NH2-GFP-tmpk-COOH). Similarly, a fusion polynucleotide can comprise a tmpk component fused to a docking polynucleotide (e.g. 5′-tmpkGFP) or alternatively can comprise a docking component fused to a tmpk molecule (e.g 5′-GFP-tmpk-3′)).

The docking molecule optionally permits isolation of tmpk expressing or therapeutic gene expressing cells. A person skilled in the art would recognize that many molecules are useful for fusing to tmpk to permit isolation of modified tmpk or control expressing cells. Choice of molecule will depend on the cell type to be transfected or transduced. Generally, the docking molecule is not expressed on the cell type to be transfected or transduced in appreciable levels permitting targeting and/or isolation of cells expressing the docking polynucleotide. In one embodiment the docking polynucleotide encodes a CD19 (SEQ ID NOS: 27-28). In a preferred embodiment, the docking polynucleotide encodes a truncated CD19 (SEQ ID NOS: 29-31). In an alternate embodiment, the detection docking polynucleotide encodes CD25. In another embodiment, the docking polynucleotide encodes a fluorescent protein such as EGFP. In another embodiment, the molecules encoded by the docking polynucleotide comprise CD20, CD25, low affinity nerve growth factor receptor (LNGFR), truncated CD34, or erythropoietin receptor (EpoR).

In addition, the tmpk and docking components are optionally discontinuous. For example a linker sequence is optionally present between the tmpk and docking components.

The term “linker sequence” as used in reference to a tmpk/docking fusion refers to residues that link the tmpk and docking components. In a polypeptide, the residues are generally amino acids. In a polynucleotide, the residues are generally nucleotides. The term “linker sequence” as used in reference to an activator/docking fusion polypeptide accordingly generally refers to a sequence of amino acids that links the activator and docking components. The term “linker sequence” as used in reference to a tmpk/docking fusion polynucleotide accordingly generally refers to a sequence of nucleotides that link the tmpk and docking components. The linker when referring to a polypeptide sequence optionally comprises 3, 4, 5, 6, 6-10, 10-15 or 15-25 amino acids or longer and when referring to a polynucleotide sequence comprises 3-6, 6-12, 18, 12-24, or 24-72 nucleic acid residues or longer. A linker sequence is useful for several reasons. A linker sequence can be used to facilitate cloning. Further a linker sequence can provide a gap between the components that facilitates proper folding and/or activity (e.g. antigenic activity for the docking component and/or catalytic activity for the tmpk component). A person skilled in the art will recognize that a number of linker sequences can be used and a number of linker sequences are known in the art. The linker sequence can comprise any sequence of amino acids or nucleotides that is suitable. For example, suitable refers to the amino acid composition of the linker. For example, uncharged amino acids are preferable. Amino acids such as proline which could limit the flexibility of the linker are generally not preferred. In one embodiment the components are comprised in a discontinuous relationship, the fusion polypeptide optionally comprises a tmpk component fused to a linker fused to a docking polypeptide (e.g. NH2-tmpk-linker-GFP) or alternatively comprises a docking polypeptide component fused to a linker fused to a tmpk molecule (e.g NH2-truncated CD19-linker-tmpk-COOH). Similarly, a fusion polynucleotide can comprise a tmpk component fused to a linker fused to a docking polynucleotide (e.g. 5′-tmpk-linker-GFP′) or alternatively can comprise a docking polynucleotide component fused to a linker fused to a tmpk molecule (e.g 5′-truncated CD19-linker-tmpk-3′; such as SEQ ID NO: 28, 29, 31 or 37 fused to a linker sequence described herein fused to SEQ ID NO:36)). The tmpk and docking components are fused in frame such that both components are expressed together as one continuous polypeptide sequence in each cell.

### Delivery Vectors

It will be appreciated by one skilled in the art that a variety of delivery vectors and expression vehicles are usefully employed to introduce a modified DNA molecule into a cell. Vectors that are useful comprise lentiviruses, oncoretroviruses, expression plasmids, adenovirus, and adeno-associated virus. Other delivery vectors that are useful comprise herpes simplex viruses, transposons, vaccinia viruses, human papilloma virus, Simian immunodeficiency viruses, HTLV, human foamy virus and variants thereof. Further vectors that are useful comprise spumaviruses, mammalian type B retroviruses, mammalian type C retroviruses, avian type C retroviruses, mammalian type D retroviruses, HTLV/BLV type retroviruses, and lentiviruses.

Vectors such as those listed above have been employed to introduce DNA molecules into cells for use in gene therapy. Examples of vectors used to express DNA in cells include: Kanazawa T, Mizukami H, Okada T, Hanazono Y, Kume A, Nishino H, Takeuchi K, Kitamura K, Ichimura K, Ozawa K. Suicide gene therapy using AAV-HSVtk/ganciclovir in combination with irradiation results in regression of human head and neck cancer xenografts in nude mice. Gene Ther. 2003 January; 10(1):51-8. Fukui T, Hayashi Y, Kagami H, Yamamoto N, Fukuhara H, Tohnai I, Ueda M, Mizuno M, Yoshida J Suicide gene therapy for human oral squamous cell carcinoma cell lines with adeno-associated virus vector. Oral Oncol. 2001 April; 37(3):211-5.

### Lentiviral Vectors

The safety facet of suicide gene therapy relies on efficient delivery and stable, consistent expression of both the therapeutic and the safety component genes. LVs transduce a wide range of dividing and non-dividing cell types with high efficiency, conferring stable, long-term expression of the transgene²⁵⁻²⁷.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

In one embodiment the Lentigen lentiviral vector described in Lu, X. et al. Journal of gene medicine (2004) 6:963-973 is used to express the DNA molecules.

In one embodiment the disclosure comprises a lentiviral vector expressing a dck or modified tmpk molecule. In one embodiment the lentiviral vector comprises a 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). It will be readily apparent to one skilled in the art that optionally one or more of these regions is substituted with another region performing a similar function.

Gene therapy requires the transgene product to be expressed at sufficiently high levels. Enhancer elements can be used to increase expression of modified DNA molecules or increase the lentiviral integration efficiency. Locus control regions or scaffold attachment regions can also be added to vectors to mitigate position-mediated expression effects or the likelihood that dysfunctional expression of surrounding genes will occur. In one embodiment the lentiviral vector further comprises a nef sequence. In a preferred embodiment the lentiviral further comprises a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. In an alternate preferred embodiment, the lentiviral vector further comprises a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to lentiviral vector results in a substantial improvement in the level of transgene expression from several different promoters, both in vitro and in vivo. In a further preferred embodiment, the lentiviral vector comprises both a cPPT sequence and WPRE sequence. The vector also comprises in an alternate embodiment an internal ribosome entry site (IRES) sequence that permits the expression of multiple polypeptides from a single promoter. In another embodiment the lentiviral vector comprises a detection cassette. In another embodiment, the detection cassette comprises a CD19 molecule or fragment thereof. In another preferred embodiment the plasmid comprises a docking polynucleotide incorporated into pHR′-cppt-EF-IRES-W-SIN, pHR′-cppt-EF-tmpk(R16GLL)-IRES-hCD19-W-SIN or pHR′-cppt-EF-tmpk(F105Y)-IRES-hCD19-W-SIN. Additionally it will be readily apparent to one skilled in the art that optionally one or more of these elements can be added or substituted with other regions performing similar functions.

In addition to IRES sequences, other elements which permit expression of multiple polypeptides are useful. In one embodiment the vector comprises multiple promoters that permit expression more than one polypeptide. In another embodiment the vector comprises a protein cleavage site that allows expression of more than one polypeptide. Examples of protein cleavage sites that allow expression of more than one polypeptide comprise those listed in the following articles which are incorporated by reference: Retroviral vector-mediated expression of HoxB4 in hematopoietic cells using a novel coexpression strategy. Klump H, Schiedlmeier B, Vogt B, Ryan M, Ostertag W, Baum C. Gene Ther. 200; 8(10):811-7; A picornaviral 2A-like sequence-based tricistronic vector allowing for high-level therapeutic gene expression coupled to a dual-reporter system Mark J. Osborn, Angela Panoskaltsis-Mortari, Ron T. McElmurry, Scott K. Bell, Dario A. A. Vignali, Martin D. Ryan, Andrew C. Wilber, R. Scott Mclvor, Jakub Tolar and Bruce R. Blazar. Molecular Therapy 2005; 12 (3), 569-574; Development of 2A peptide-based strategies in the design of multicistronic vectors. Szymczak A L, Vignali D A. Expert Opin Biol Ther. 2005; 5(5):627-38; Correction of multi-gene deficiency in vivo using a single ‘self-cleaving’ 2A peptide-based retroviral vector. Szymczak A L, Workman C J, Wang Y, Vignali K M, Dilioglou S, Vanin E F, Vignali D A. Nat Biotechnol. 2004; 22(5):589-94. It will be readily apparent to one skilled in the art that other elements that permit expression of multiple polypeptides are useful and readily utilized in the vectors of the disclosure.

### Viral Regulatory Elements

In addition to the viral regulatory elements described above, additional viral regulatory elements are readily included in the vector constructs of the application. Viral regulatory elements are components of vehicles used to introduce nucleic acid molecules into a host cell. The viral regulatory elements are optionally retroviral regulatory elements. For example, the viral regulatory elements may be the LTR and gag sequences from HIV1, HSC1, or MSCV. The retroviral regulatory elements may be from lentiviruses or they may be heterologous sequences identified from genomic regions.

One skilled in the art would also appreciate that as other viral regulatory elements are identified, these may be used with the nucleic acid molecules of the disclosure.

### Detection or Selection Cassette

As noted above, the docking polynucleotide produces a docking polypeptide, such as a cell surface polypeptide (eg. CD19, CD20 or CD25), that is recognized by a toxic binding agent. An example of a toxic binding agent is an antibody such as Herceptin or an antibody conjugated to a toxin. The docking polypeptide when a cell surface polypeptide is optionally used as a detection and/or selection cassette is also referred to as a detection or selection marker. In other embodiments the vector construct comprises a detection or selection marker that is distinct from the docking polypeptide.

In suicide gene therapy, it is typically desirable that the majority of transduced cells express the suicide gene or genes. This need can be met by co-introducing a detection marker polynucleotide, which in some cases can be the same gene as the docking polynucleotide. In other cases, the detection marker polynucleotide is different than the docking polypeptide recognized by the toxic binding agent. Transduced cells are readily identified and enriched based on expression of this detection marker polynucleotide. A good detection marker should be inert in itself, devoid of signaling capacity and non-immunogenic. A variety of detection markers can be used in this context: human CD24, murine HSA, human CD25 (huCD25) and a truncated form of LNGFR.

A novel truncated form of CD19 (CD19Δ) is optionally adopted as a detection marker (SEQ ID NOS: 29-31). CD19 (SEQ ID NOS: 27-28) is a 95-kDa glycoprotein of the immunoglobulin superfamily. It forms a complex with CD21, CD81, and Leu-13, and collectively functions to modulate the activation threshold of the B cell receptor. As expression of CD19 and CD21 is restricted to B cell lineages from immature progenitors to blasts, it is suitable for use in murine and human T cells. To further decrease any signaling capacity from the CD19 molecule, the cytoplasmic tail has been deleted for the present adaptation. In one embodiment truncated CD19 comprises all or a portion of SEQ ID NO: 29. In another embodiment truncated CD19 comprises all or a portion of SEQ ID NO: 30. In another embodiment truncated CD19 comprises all or a portion of SEQ ID NO: 31.

“Detection cassette” or “detection marker” is used to refer to a polynucleotide that directs expression of a molecule that acts as a selection marker and that optionally provides for a mode of isolating cells expressing said selection marker. The molecule is optionally used to select transduced or transfected cells or to determine the efficiency of cell transduction or transfection. Molecules that are useful as selection markers or detection agents comprise CD19, truncated CD19, CD25 and EGFP. EGFP is variably referred to as enGFP herein. One skilled in the art will recognize that other fluorescent molecules are similarly used.

As mentioned, in certain embodiments, the detection cassette is also a docking gene. In certain embodiments, the detection cassette and docking gene are different.

As mentioned, the detection cassette encodes a selection molecule that is typically used to isolate transduced or transfected cells. The detection cassette is useful in vectors comprising therapeutic gene and/or an activator polynucleotide or control molecules. Control molecules include molecules that do not function as suicide gene therapy molecules that are typically employed to assess the effect of mutants in similarly related cells. A person skilled in the art would recognize that many molecules are useful to permit isolation of cells. Choice of molecule will depend on the cell type to be transfected or transduced. The detection cassette molecule is not expressed on the cell type to be transfected or transduced in appreciable levels permitting isolation of cells expressing the detection cassette. In one embodiment the detection cassette encodes a CD19 (SEQ ID NOS: 27-28) selection marker. In a preferred embodiment, the detection cassette encodes a truncated CD19 (SEQ ID NOS: 29-31) selection marker. In an alternate embodiment, the detection cassette encodes CD25. In another embodiment, the detection cassette encodes a fluorescent protein such as EGFP. In another embodiment, the molecules encoded by the detection cassette comprise CD20, CD25, low affinity nerve growth factor receptor (LNGFR), truncated CD34, or erythropoietin receptor (EpoR). Additionally, the detection cassette optionally comprises a drug resistance gene permitting isolation of transduced or transfected cells by drug selection.

### Polynucleotides of Interest/Therapeutic Nucleic Acid Molecules

Cells transfected or transduced in vitro with the vector constructs described herein are useful for ex vivo gene therapy or as a research tool or for protein production. Nucleic acid molecules described herein are also useful for gene therapy by transfecting or transducing cells in vivo to express a therapeutic polynucleotide/protein in addition to activator polynucleotide and docking polynucleotide. The therapeutic polynucleotide is alternatively referred to herein as the therapeutic gene, therapeutic cassette and/or therapeutic expression cassette. For example, if one were to upregulate the expression of a gene, one could insert the sense polynucleotide into a vector construct described herein. If one were to downregulate the expression of the gene, one could insert the antisense or an siRNA polynucleotide sequence into the therapeutic expression cassette. Techniques for inserting sense and antisense sequences (or fragments of these sequences) would be apparent to those skilled in the art. The therapeutic nucleic acid molecule or nucleic acid molecule fragment is optionally either isolated from a native source (in sense or antisense orientations) or synthesized. It is also optionally a mutated native or synthetic sequence or a combination of these.

Examples of therapeutic coding nucleic acid molecules to be expressed include adenosine deaminase (ADA), γc interleukin receptor subunit, α-galactosidase A (α-galA), acid ceramidase, galactocerebrosidase, and transmembrane conductance regulator (CFTR) molecules.

Other molecules may also be introduced. For example T cells may be genetically modified to express other relevant molecules for therapy such as T cell receptors. Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mavroukakis S A, Rosenberg S A. Cancer regression in patients after transfer of genetically engineered lymphocytes. Science. 2006 October 6; 314(5796):126-9. Epub 2006 Aug. 31.PMID: 16946036 [PubMed—indexed for MEDLINE]

### Pharmaceutical Compositions

Another aspect relates to pharmaceutical compositions comprising the vector constructs described herein for use in a system comprising a corresponding prodrug and/or toxic binding agent. The pharmaceutical compositions of this disclosure are used to treat patients having diseases, disorders or abnormal physical states could include an acceptable carrier, auxiliary or excipient.

The pharmaceutical compositions are optionally administered by ex vivo and in vivo methods such as electroporation, DNA microinjection, liposome DNA delivery, and virus vectors that have RNA or DNA genomes including retrovirus vectors, lentivirus vectors, Adenovirus vectors and Adeno-associated virus (AAV) vectors, Semliki Forest Virus, Vaccinia virus, Herpes Simplex Virus, Vesticular Stomatitis Virus, etc. Derivatives or hybrids of these vectors are also useful.

Dosages to be administered depend on patient needs, on the desired effect and on the chosen route of administration. The expression cassettes are optionally introduced into the cells or their precursors using ex vivo or in vivo delivery vehicles such as liposomes or DNA or RNA virus vectors. They are also optionally introduced into these cells using physical techniques such as microinjection or chemical methods such as coprecipitation.

The pharmaceutical compositions are typically prepared by known methods for the preparation of pharmaceutically acceptable compositions which are administered to patients, and such that an effective quantity of the nucleic acid molecule is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA).

On this basis, the pharmaceutical compositions could include an active compound or substance, such as a nucleic acid molecule, in association with one or more pharmaceutically acceptable vehicles or diluents, and contained in buffered solutions with a suitable pH and iso-osmotic with the physiological fluids. The methods of combining the expression cassettes with the vehicles or combining them with diluents is well known to those skilled in the art. The composition could include a targeting agent for the transport of the active compound to specified sites within cells.

The application also provides compositions comprising the prodrug and/or toxic binding agent for use with the safety gene therapy system described herein. In the event of an adverse transplant or gene therapy related event, or any situation where it is desirable to clear cells modified to express the activator and docking genes of the disclosure, a subject, preferably a human patient is administered a composition comprising a suitable prodrug and/or toxic binding agent. The prodrug can be administered contemporaneously with a composition comprising a toxic binding agent. In other embodiments, the prodrug and toxic binding agent are administered separately.

The term “treating” or “treatment” as used herein means administering to a subject a therapeutically effective amount of the compound of the present application and may consist of a single administration, or alternatively comprise a series of applications. For example, the compound of the present application may be administered at least once a week. However, in another embodiment, the compound may be administered to the subject from about one time per week to about once daily for a given treatment. The length of the treatment period depends on a variety of factors, such as the severity of the disease, the age of the patient, the concentration and the activity of the compounds of the present application, or a combination thereof. In one embodiment, the treatment is chronic treatment and the length of treatment is 1-2 weeks, 2-4 weeks or more than 4 weeks. The treatment regimen can include repeated treatment schedules. It will also be appreciated that the effective amount or dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

As used herein, and as well understood in the art, “treatment” or “treating” is also an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Further any of the treatment methods or uses described herein can be formulated alone or for contemporaneous administration with other agents or therapies.

As used herein, the phrase “effective amount” or “therapeutically effective amount” or a “sufficient amount” of a compound or composition of the present application is a quantity sufficient to, when administered to the subject, including a mammal, for example a human, effect beneficial or desired results, including clinical results, and, as such, an “effective amount” or synonym thereto depends upon the context in which it is being applied. For example, in the context of treating GVHD, it is an amount of the compound sufficient to achieve a treatment response as compared to the response obtained without administration of the compound. The amount of a given compound of the present application that will correspond to such an amount will vary depending upon various factors, such as the given drug or compound, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a compound of the present disclosure is an amount which results in a beneficial or desired result in a subject as compared to a control. As defined herein, a therapeutically effective amount of a compound of the present disclosure may be readily determined by one of ordinary skill by routine methods known in the art. Dosage regime may be adjusted to provide the optimum therapeutic response.

The term “subject” as used herein includes all members of the animal kingdom including mammals, suitably humans including patients.

Compositions comprising the prodrug and/or toxic binding agent can be administered by various routes. For example, oral formulations of AZT are well known in the art. Accordingly the prodrug can be administered orally. The toxic binding agent can be administered in one embodiment intraperitoneally (i.p.), intravenously (i.v.) or intratumorally,

In other embodiments, the composition comprises cells modified with the vector constructs described herein. Such modified cells can be administered intravenously using methods known in the art i.p., i.v., intratumorally, stereotactic injections to a variety of sites, direct injections, intramuscularly, etc.

### Host Cells

The disclosure also relates to a host cell (isolated cell in vitro, a cell in vivo, or a cell treated ex vivo and returned to an in vivo site) containing a nucleic acid molecule of the disclosure. Cells transfected with a nucleic acid molecule such as a DNA molecule, or transduced with the nucleic acid molecule such as a DNA or RNA virus vector, are optionally used, for example, in bone marrow or cord blood cell transplants according to techniques known in the art. Examples of the use of transduced bone marrow or cord blood cells in transplants are for ex vivo gene therapy of Adenosine deaminase (ADA) deficiency. Other cells which are optionally transfected or transduced either ex vivo or in vivo include purified stem cells (of embryonic or later ontogeny), as described above.

### Methods of Isolation

In one aspect of the present disclosure, methods for expressing a vector construct of the disclosure in cells for transplant are provided. After transduction or transfection with vectors comprising elements such as the activator polynucleotide and docking/selection polynucleotide, cells expressing these molecules are optionally isolated by a variety of means known in the art. In certain embodiments, the cells are isolated by cell sorting or flow cytometry using an antibody to the detection cassette encoded selection marker. Additionally cell sorting is useful to isolate modified cells where the detection cassette is a fluorescent protein such as EGFP. Cells expressing polynucleotides of the disclosure are, in an alternate embodiment, isolated using magnetic sorting or other immuno-selection schemas. Additionally, cells may be isolated by drug selection. In one embodiment, a vector comprising a drug resistance gene and a polynucleotides of the disclosure is introduced into cells. Examples of drug resistance genes include, but are not limited to, neomycin resistance gene, blasticidin resistance gene (Bsr), hygromycin resistance gene (Hph), puromycin resistance gene (Pac), Zeocin resistance gene (Sh ble), FHT, bleomycin resistance gene and ampicillin resistance gene. After transduction or transfection, modified cells including the drug resistance gene are selected by adding the drug that is inactivated by the drug resistance gene. Cells expressing the drug resistance gene survive while non-transfected or non-transduced cells are killed. A person skilled in the art would be familiar with the methods and reagents required to isolate cells expressing the desired polynucleotides.

### Cell Types for Transplant

Compositions and vector constructs of the disclosure are usefully introduced into any cell type ex vivo where it is desirable to provide a mechanism for killing the modified cells. Cell types that are useful in one embodiment of the present disclosure include, but are not limited to, stem cells (both embryonic and of later ontogeny), cord blood cells, and immune cells such as T cells, adherent and non-adherent bone marrow cells and peripheral blood mononuclear cells including dendritic cells. T-cells are optionally CD4 positive, CD8 positive, CD4/CD8 double positive, or CD4/CD8 double negative. These latter cells are useful for inducing tolerance. In addition, T cells are optionally mature T cells. In one embodiment T cells are transduced with a vector of the disclosure, isolated and transplanted in a host. In another embodiment the T cells are mature T cells. In an alternate embodiment stem cells are transduced, isolated and transplanted in a host.

Cell lines are optionally transduced. For example human T cell leukemia Jurkat T cells, human erythro-leukemic K562 cells, human prostate cell lines DU145 and PC3 cells are optionally transduced or transfected with polynucleotides of the disclosure. Primary human tumor cells can also be isolated and transduced by this method. With addition of other modulator polypeptides such as IL-12, these cells can be made to become potent initiators of anti-leukemia responses. Endowment of such cells with a suicide mechanism will allow their selective removal after anti-tumor immune response initiation. Such selective killing and engulfment of dying cells by antigen-presenting cells can serve to augment the specific anti-tumor response.

### Methods of Treatment

The present disclosure provides modified compositions and vector constructs for treatment of diseases such as Fabry and Farber diseases. The compositions and vectors are also useful for the reduction of cell proliferation, for example for treatment of cancer. The present disclosure also provides methods of using compositions and vectors of the disclosure for expressing therapeutic polynucleotides for the reduction of cell proliferation, for example for treatment of cancer.

Vector constructs are introduced into cells that are used for transplant or introduced directly in vivo in mammals, preferably a human. The vector constructs are typically introduced into cells ex vivo using methods known in the art. Methods for introducing vector constructs comprise transduction, transfection, infection, electroporation. These methods optionally employ liposomes or liposome like compounds.

In one embodiment, compositions and vectors of the disclosure are used to treat cancer by adoptive therapy. Adoptive therapy or adoptive (immuno)therapy refers to the passive transfer of immunologically competent tumor-reactive cells into the tumor-bearing host to, directly or indirectly, mediate tumor regression. The feasibility of adoptive (immuno)therapy of cancer is based on two fundamental observations. The first of these observations is that tumor cells express unique antigens that can elicit an immune response within the syngeneic (genetically identical or similar especially with respect to antigens or immunological reactions) host. The other is that the immune rejection of established tumors can be mediated by the adoptive transfer of appropriately sensitized lymphoid cells. Clinical applications include transfer of peripheral blood stem cells following non-myeloablative chemotherapy with or without radiation in patients with lymphomas, leukemias, and solid tumors.

In one aspect of the present disclosure, donor T cells or stem cells (either embryonic or of later ontogeny) are transduced with vector constructs of the disclosure. Cells expressing these vector constructs are isolated and adoptively transferred to a host in need of treatment. In one embodiment the bone marrow of the recipient is T-cell depleted. Methods of adoptive T-cell transfer are known in the art (J Translational Medicine, 2005 3(17): doi; 0.1186/1479-5876-3-17, Adoptive T cell therapy: Addressing challenges in cancer immunotherapy. Cassian Yee). This method is used to treat solid tumors and does not require targeting the vector construct-transduced expressing T-cells to the tumor since the modified T-cells will recognize the different MHC class molecules present in the recipient host resulting in cytotoxic killing of tumor cells.

Another aspect of the disclosure provides for the treatment of solid tumors by injecting activator polynucleotides and docking polynucleotides of the disclosure and/or vector constructs or compositions comprising the same, directly into the tumor. Methods of introducing polynucleotides of the disclosure directly in vivo in a mammal, preferably a human, comprise direct viral delivery, microinjection, in vivo electroporation, and liposome mediated methods.

Activator genes have been introduced by injection directly into the site of a tumor to examine results of the technique as a cancer therapeutic treatment (Chevez-Barrios P, Chintagumpala M, Mieler W, Paysse E, Boniuk M, Kozinetz C, Hurwitz M Y, Hurwitz R L. Response of retinoblastoma with vitreous tumor seeding to adenovirus-mediated delivery of thymidine kinase followed by ganciclovir. J Clin Oncol. 2005 November 1; 23(31):7927-35. Sterman D H, Treat J, Litzky L A, Amin K M, Coonrod L, Molnar-Kimber K, Recio A, Knox L, Wilson J M, Albelda S M, Kaiser L R. Adenovirus-mediated herpes simplex virus thymidine kinase/ganciclovir gene therapy in patients with localized malignancy: results of a phase I clinical trial in malignant mesothelioma. Hum Gene Ther. 1998 May 1; 9(7):1083-92). The activator polynucleotides and docking polynucleotides of the present disclosure are optionally introduced directly into the site of a tumor to reduce proliferation of tumor cells, for example, to treat cancer.

In one embodiment, cells are transfected or transduced ex vivo with vectors. In an optional embodiment, the vector comprises a lentiviral vector.

### Tissue Specific Expression

In an alternate embodiment of the disclosure, the modified expressing cells express activator polynucleotides and docking polynucleotides under the control of a tissue or cell specific promoter providing expression in a tissue specific manner. Expression of modified activator polynucleotides and docking polynucleotides is optionally targeted to tumor cells using promoters that are active in tumor cells.

Accordingly, in one aspect of the disclosure, delivery vectors comprising activator polynucleotides and docking polynucleotides molecules are provided that result in tissue or cell specific expression. Tissue and cell specific expression is typically accomplished using promoters operably linked with the activator polynucleotides and docking polynucleotides, which limit expression to cells or tissues. One skilled in the art will recognize that a variety of promoter sequences that direct tissue or cell specific expression are useful to direct tissue or cell specific expression. For example, one skilled in the art will readily recognize that liver specific expression is accomplished using a liver specific promoter. Expression is readily limited to a variety of cell and tissue types. Examples include, but are not limited to, liver, heart, pancreas and T cells. Examples of liver specific promoters include, but are not limited to, the transthyretin promoter, albumin promoter, alpha feto protein promoter. Examples of other cell specific promoters include, but are not limited to, islet cell specific promoters such as the insulin promoter, and T cell specific promoters such as CD4-promoter. In another embodiment, expression is inducible. The hypoxia-inducible promoter is optionally used to direct expression of a cytoprotective gene such as but not limited to erythropoietin. Introduction of a cytoprotective gene under the control of a inducible promoter such as the hypoxia inducible promoter is useful, to prevent the severe tissue damage by hypoxia. Other promoters are also useful. For example, tet regulator inducible systems can be employed including tet on and tet off versions. Other analogous activating systems are known in the artlf the transduced cells cause some problems, the transduced cells are optionally cleared (killed) by suicide effect by administering prodrug and/or toxic binding agent to the transduced cells.

Tumor cell specific expression is accomplished using a tumor specific promoter. Tumor specific promoters comprise the progression elevated gene-3 (PEG-3) promoter. This promoter functions selectively in divergence cancer cells with limited activity in normal cells, for tumor cell-specific expression. Other tumor specific promoters are also known in the art. The transduced tumor cells are specifically killed by the prodrug and toxic binding agent.

### Graft Versus Leukemia

In addition, the disclosure provides, in one aspect, a method of treating leukemia. Donor T cells or stem cells are transduced with vectors comprising activator polynucleotides and docking polynucleotides, cells expressing said activator polynucleotides and docking polynucleotides are isolated and transplanted to a host in need of treatment. The transplanted cells induce a graft versus leukemia effect. If the transplanted cells induce graft versus host disease, the transplanted cells can be killed by administering a prodrug or toxic binding agent.

Graft versus leukemia refers to using donor transplant cells to kill host leukemic cells. Introduced cells will often also attack the cancer cells that still may be present after transplant. This was first documented in acute leukemia, and this phenomenon has been called “graft-versus-leukemia” effect. Similar effects have been observed in malignant lymphoma, myeloma, and even some solid tumors. For certain diseases, such as chronic myelogenous leukemia (CML), the graft-versus-leukemia (GvL) effect may well be the most important reason that allogeneic transplants are successful in curing the disease.

### Graft Versus Host Disease (GVHD)

The infusion of donor lymphocytes in allogenic bone marrow transplant (BMT) recipients provides potent antitumor activity to treat recurrent malignancies. One complication, however, is severe Graft Versus Host Disease (GVHD).

Graft versus host disease is a common complication of allogeneic bone marrow transplantation (BMT). After bone marrow transplantation, T cells present in the graft, either as contaminants or intentionally introduced into the host, attack the tissues of the transplant recipient. Graft-versus-host disease can occur even when HLA-identical siblings are the donors. HLA-identical siblings or HLA-identical unrelated donors (called a minor mismatch as opposed to differences in the HLA antigens, which constitute a major mismatch) often still have genetically different proteins that can be presented on the MHC.

Graft versus host disease is a serious complication of transplant and can lead to death in patients that develop severe graft versus host disease (the clinical manifestations of graft versus host disease are reviewed in Socie G. Chronic graft-versus-host disease: clinical features and grading systems. Int J Hematol. 2004 April; 79(3):216-20). Viral thymidine kinase has been introduced into transplant cells and used in combination with drugs such as ganciclovir to determine the results in individuals who develop graft versus host disease. (Bonini C, Ferrari G, Verzeletti S, Servida P, Zappone E, Ruggieri L, Ponzoni M, Rossini S, Mavilio F, Traversari C, Bordignon C HSV-TK gene transfer into donor lymphocytes for control of allogeneic graft-versus-leukemia. Science. 1997 June 13; 276(5319):1719-24; Bondanza A, Valtolina V, Magnani Z, Ponzoni M, Fleischhauer K, Bonyhadi M, Traversari C, Sanvito F, Toma S, Radrizzani M, La Seta-Catamancio S, Ciceri F, Bordignon C, Bonini C Suicide gene therapy of graft-versus-host disease induced by central memory human T lymphocytes. Blood. 2005.)

While donor T-cells are undesirable as effector cells of graft-versus-host-disease, they are valuable for engraftment by preventing the recipient's residual immune system from rejecting the bone marrow graft (host-versus-graft). Additionally, as bone marrow transplantation is frequently used to cure malignant disorders (most prominently the leukemias), donor T-cells have proven to have a valuable graft-versus-tumor (GVT, graft versus leukemia described above) effect. A great deal of current research on allogeneic bone marrow transplantation involves attempts to separate the undesirable graft-vs-host-disease aspects of T-cell physiology from the desirable graft-versus-tumor effect.

The present disclosure provides, in one embodiment, methods of treating transplant patients that develop graft versus host disease by administering compounds described herein (e.g. activator polynucleotides and docking polynucleotides used in combination with drugs and/or toxic binding agents) to a mammal in need thereof. For example, transplant cells modified to express polypeptides encoded by activator polynucleotides and docking polynucleotides are treated with prodrug and/or a toxic binding agent to clear the modified cells. In another embodiment, the disclosure provides a method of promoting graft versus tumor effect by administering compounds of the disclosure to a mammal in need thereof.

Vector constructs containing the nucleic acid molecules of the disclosure are typically administered to mammals, preferably humans, in gene therapy using techniques described below. The polypeptides produced from the nucleic acid molecules are also optionally administered to mammals, preferably humans. The disclosure relates to a method of medical treatment of a mammal in need thereof, preferably a human, by administering to the mammal a vector of the disclosure or a cell containing a vector of the disclosure. A recipient, preferably human, who develops an adverse event, such as graft versus host disease, is typically administered a drug, such as AZT, that is a substrate for the polypeptide produced by the activator polynucleotides of the disclosure. The subject is also optionally administered a toxic binding agent, such as an antibody conjugated t a toxin. Diseases, such as blood diseases or neural diseases (neurodegenerative), that are readily treated are described in this application and known in the art (eg. diseases, such as thalassemia or sickle cell anemia that are treated by administering a globin gene as described in Canadian patent application no. 2,246,005). Blood diseases treatable by stem cell transplant include leukemias, myelodysplastic syndromes, stem cell disorders, myeloproliferative disorders, lymphoproliferative disorders phagocyte disorders, inherited metabolic disorders, histiocytic disorders, inherited erythrocyte abnormalities, inherited immune system disorders, inherited platelet abnormalities, plasma cell disorders, malignancies (See also, Medical Professional's Guide to Unrelated Donor Stem Cell Transplants, 4th Edition). Stem cell nerve diseases either inherited or acquired to be treated by neural stem cell transplantation include diseases resulting in neural cell damage or loss, eg. paralysis, Parkinson's disease, Alzheimer's disease, ALS, multiple sclerosis, traumatic injury). The vector constructs of the disclosure are useful for providing a stem cell marker and to express genes that cause stem cells to differentiate (e.g. growth factor).

The inventors have achieved long-term enzymatic correction and corresponding lipid reduction in a mouse model of Fabry disease by bone marrow transplantation (BMT) of transduced cells9-11 and by direct delivery of lentivirus into neonates.12

The inventors have developed and utilized retroviral vectors that engineer expression of both α-gal A and human CD25 (huCD25) in a bicistronic format.13 CD25, also known as the T-cell activation antigen (Tac) and the interleukin (IL)-2 receptor alpha chain-α,14 is incapable of mediating IL-2 internalization or signaling by itself; however, in tandem with the β-chain of the receptor and the γcchain, it forms the “high-affinity” receptor for IL-2.15 Though it can be induced upon activation, expression of CD25 is absent on resting T cells, B cells, monocytes, and CD34+-enriched cells.16,17 Thus, its limited expression pattern and lack of ability to mediate signaling make it a good choice as a cell surface marking protein in bicistronic vectors. In previous studies, huCD25 expression was used functionally to assess viral titers, for the enrichment of transgene-positive cells before BMT, and for tracking transduced cells after BMT.13 As it is cleaved from the IL-2 receptor complex on the cell surface and can be detected as soluble CD25 (sCD25) in the plasma,18 sCD25 was also used as a surrogate marker to evaluate the level of transgene expression in an experimental setting.12

The inventors now extend the use of huCD25 expression from bicistronic retroviral vector constructs into the development and application of a built-in safety mechanism within the gene therapy context. Unwanted proliferative abnormality occurs following retroviral gene transfer, huCD25 can act as a target antigen to eliminate transduced cells selectively using either clinically approved anti-CD25 antibodies or newer, highly potent antibody-toxin conjugates (immunotoxins).

Using a murine leukemia model, the inventors demonstrated that antibody treatment reduced tumor burden 32-fold and increased survival compared with untreated mice. Furthermore, after a bone marrow transplant of therapeutically transduced cells into Fabry mice, antibody treatment reduced the number of retrovirally transduced huCD25-expressing cells in the peripheral blood. A systemic loss of transduced cells with functional consequences was also evident in the liver and spleen.

### Gene Therapy

The disclosure includes compositions and methods for providing a coding nucleic acid molecule or therapeutic gene to a subject such that expression of the molecule in the cells provides the biological activity of the polypeptide encoded by the coding nucleic acid molecule to those cells. A coding nucleic acid as used herein means a nucleic acid that comprises nucleotides which specify the amino acid sequence, or a portion thereof, of the corresponding protein. A coding sequence may comprise a start codon and/or a termination sequence.

The disclosure includes methods and compositions for providing a coding nucleic acid molecule to the cells of an individual such that expression of the coding nucleic acid molecule in the cells provides the biological activity or phenotype of the polypeptide encoded by the coding nucleic acid molecule. The method also relates to a method for providing an individual having a disease, disorder or abnormal physical state with a biologically active polypeptide by administering a nucleic acid molecule of the present disclosure. The method may be performed ex vivo or in vivo. Gene therapy methods and compositions are demonstrated, for example, in U.S. Pat. Nos. 5,869,040, 5,639,642, 5,928,214, 5,911,983, 5,830,880,5,910,488, 5,854,019, 5,672,344, 5,645,829, 5,741,486, 5,656,465, 5,547,932, 5,529,774, 5,436,146, 5,399,346 and 5,670,488, 5,240,846. The amount of polypeptide will vary with the subject's needs. The optimal dosage of vector may be readily determined using empirical techniques, for example by escalating doses (see U.S. Pat. No. 5,910,488 for an example of escalating doses).

Various approaches to gene therapy may be used. The disclosure includes a process for providing a human with a therapeutic polypeptide including: introducing human cells into a human, said human cells having been treated in vitro or ex vivo to insert therein a vector of the disclosure, the human cells expressing in vivo in said human a therapeutically effective amount of said therapeutic polypeptide.

The method also relates to a method for producing a stock of recombinant virus by producing virus suitable for gene therapy comprising modified DNA encoding a gene of interest. This method preferably involves transfecting cells permissive for virus replication (the virus containing therapeutic gene) and collecting the virus produced.

Cotransfection (DNA and marker on separate molecules) may be employed (see eg U.S. Pat. No. 5,928,914 and U.S. Pat. No. 5,817,492). As well, a detection cassette or marker (such as Green Fluorescent Protein marker or a derivative) may be used within the vector itself (preferably a viral vector).

### Polypeptide Production and Research Tools

A cell line (either an immortalized cell culture or a stem cell culture) transfected or transduced with a polynucleotide of the disclosure (or variants) is useful as a research tool to measure levels of expression of the coding nucleic acid molecule and the activity of the polypeptide encoded by the coding nucleic acid molecule.

The disclosure includes a method for producing a recombinant host cell capable of expressing a nucleic acid molecule of the disclosure comprising introducing into the host cell a vector of the disclosure.

The disclosure also includes a method for expressing a polypeptide in a host cell of the disclosure including culturing the host cell under conditions suitable for coding nucleic acid molecule expression. The method typically provides the phenotype of the polypeptide to the cell.

In these methods, the host cell is optionally a stem cell or a T cell.

Another aspect of the disclosure is an isolated polypeptide produced from a nucleic acid molecule or vector of the disclosure according to a method of the disclosure.

### Uses

The application further provides various uses of the safety system and vector constructs described herein.

### Uses of Activator/Docking Polynucleotide

Also provided are a number of uses of suicide gene systems comprising activator and docking polynucleotides and activator/docking genes. All the aforementioned activator polynucleotides, tmpk variants, delivery vectors, docking polynucleotides, therapeutic genes, methods and composition embodiments are contemplated for the various uses herein described.

One embodiment provides use of a suicide gene system comprising a vector construct comprising a stably integrating delivery vector; an activator polynucleotide such as a modified mammalian thymidylate kinase (tmpk) polynucleotide; and a docking polynucleotide wherein the docking polynucleotide encodes a docking polypeptide, such as truncated CD19 for expressing an activator polynucleotide such as a modified mammalian tmpk polynucleotide in a mammalian cell or subject. In one embodiment the truncated CD19 is fused to the tmpk polynucleotide. In certain embodiments, the vector construct further comprises a therapeutic gene and the suicide gene system is for expressing the therapeutic gene. The suicide cell system further comprises use of a prodrug such as AZT that is converted to a drug by a polypeptide encoded by an activator polynucleotide and/or a toxic binding polypeptide that binds the docking polypeptide.

Another embodiment provides a suicide gene system comprising a vector construct comprising a stably integrating delivery vector; an activator polynucleotide such as modified mammalian thymidylate kinase (tmpk) polynucleotide; and a docking polynucleotide wherein the docking polynucleotide encodes a docking polypeptide, for expressing a modified mammalian tmpk polynucleotide and a docking polynucleotide in a mammalian cell or subject. In one embodiment, the docking polynucleotide is fused to the tmpk polynucleotide. In certain embodiments, the vector construct further comprises a therapeutic gene and the suicide gene system is for expressing the therapeutic gene. The suicide gene system further comprises use of a prodrug such as AZT that is converted to a drug by a polypeptide encoded by an activator polynucleotide or a toxic binding polypeptide that binds the docking polypeptide.

A further embodiment provides use of a suicide gene system comprising a vector construct comprising a stably integrating delivery vector; an activator polynucleotide such as modified mammalian thymidylate kinase (tmpk) polynucleotide; and a docking polynucleotide wherein the docking polynucleotide encodes a docking polypeptide, in the manufacture of a medicament for expressing a modified mammalian tmpk polynucleotide in a mammalian cell or subject. In one embodiment, the docking polynucleotide is fused to the tmpk polynucleotide.

Another embodiment provides use of a composition comprising a stably integrating delivery vector; an activator polynucleotide such as modified mammalian thymidylate kinase (tmpk) polynucleotide; and a docking polynucleotide wherein the docking polynucleotide encodes a docking polypeptide, for expressing a modified mammalian tmpk polynucleotide in a mammalian cell or subject. In one embodiment, the docking polynucleotide is fused to the tmpk polynucleotide.

A further embodiment provides a composition comprising a stably integrating delivery vector; an activator polynucleotide such as a modified mammalian thymidylate kinase (tmpk) polynucleotide; and a docking polynucleotide wherein the docking polynucleotide encodes a docking polypeptide, for expressing a modified mammalian tmpk polynucleotide in a mammalian cell or subject. In one embodiment, the docking polynucleotide is fused to the tmpk polynucleotide

Yet a further embodiment provides use of a composition comprising a stably integrating delivery vector; an activator polynucleotide such as a modified mammalian thymidylate kinase (tmpk) polynucleotide; and a docking polynucleotide wherein the docking polynucleotide encodes a docking polypeptide in the manufacture of a medicament for expressing a modified mammalian tmpk polynucleotide in a mammalian cell or subject. In one embodiment, the docking polynucleotide is fused to the tmpk polynucleotide.

Another embodiment, provides use of a vector construct or composition comprising a stably integrating delivery vector; an activator polynucleotide such as modified mammalian thymidylate kinase (tmpk) polynucleotide; and a docking polynucleotide wherein the docking polynucleotide encodes a docking polypeptide, for gene therapy. In one embodiment, the docking polynucleotide is fused to the tmpk polynucleotide.

A further embodiment provides a vector construct or composition comprising a stably integrating delivery vector; an activator polynucleotide such as a modified mammalian thymidylate kinase (tmpk) polynucleotide; and a docking polynucleotide wherein the docking polynucleotide encodes a docking polypeptide, for gene therapy. In one embodiment, the docking polynucleotide is fused to the tmpk polynucleotide.

Yet a further embodiment provides a vector construct or composition comprising a stably integrating delivery vector; an activator polynucleotide such as a modified mammalian thymidylate kinase (tmpk) polynucleotide; and a docking polynucleotide wherein the docking polynucleotide encodes a docking polypeptide, and wherein the docking polynucleotide is fused to the tmpk polynucleotide for the manufacture of a medicament for gene therapy.

Also provided is use of a vector construct or composition disclosed herein for treating a disease selected from the group consisting of cancer, GVHD or diseases resulting from a deficiency of a gene product.

Another embodiment provides a vector construct or composition disclosed herein for treating a disease selected from the group consisting of cancer, GVHD or diseases resulting from a deficiency of a gene product.

A further embodiment provides a vector construct or composition disclosed herein for the manufacture of a medicament for treating a disease selected from the group consisting of cancer, GVHD or diseases resulting from a deficiency of a gene product.

Another aspect provides use of an effective amount of a prodrug for killing a cell expressing a modified mammalian tmpk polynucleotide wherein the expression of the modified mammalian tmpk results from contact with a vector construct or composition comprising a stably integrating delivery vector; an activator polynucleotide such as a modified mammalian thymidylate kinase (tmpk) polynucleotide; and a docking polynucleotide wherein the docking polynucleotide encodes a docking polypeptide In one embodiment, the docking polynucleotide is fused to the tmpk polynucleotide.

One embodiment provides an effective amount of a prodrug for killing a cell expressing a modified mammalian tmpk polynucleotide wherein the expression of the modified mammalian tmpk results from contact with a vector construct or composition comprising a stably integrating delivery vector; an activator polynucleotide such as a modified mammalian thymidylate kinase (tmpk) polynucleotide; and a docking polynucleotide wherein the docking polynucleotide encodes a docking polypeptide. In one embodiment, the docking polynucleotide is fused to the tmpk polynucleotide.

Yet another embodiment provides use of an effective amount of a prodrug for the manufacture of a medicament for killing a cell expressing a modified mammalian tmpk polynucleotide wherein the expression of the modified mammalian tmpk results from contact with a vector construct or composition comprising a stably integrating delivery vector; an activator polynucleotide such as a modified mammalian thymidylate kinase (tmpk) polynucleotide; and a docking polynucleotide wherein the docking polynucleotide encodes a docking polypeptide. In one embodiment the docking polynucleotide is fused to the tmpk polynucleotide.

The following non-limiting examples are illustrative of the present disclosure:

### EXAMPLES

### Example 1

### Materials Methods

### Cells Lines.

The cell lines C1498 (C57BL/6 derived), 293T, 3T3, and HeLa cells (American Type Culture Collection, Manassas, Va.) were maintained in Dulbecco's modified Eagle's medium supplemented with 10% fetal calf serum (Cansera, Rexdale, Ontario), 2 mM I-glutamine, 1 mM sodium pyruvate, 100 U/ml penicillin, and 100 mg/ml streptomycin (all from Sigma, Oakville, Ontario) at 37° C. in a humidified incubator with 5% CO2.

### Vector Constructs and Viral Vector Production.

The lentiviral vector pHR′cppt-EF-α-gal A-IRES-huCD25-W-SIN (LV/α-gal A/huCD25) was constructed.12 Virus was produced by co-transfection of the lentiviral vector with accessory plasmids pMD.G and pCMVΔR8.91 into 293T cells using FuGENE 6 transfection reagent (Roche, Mississauga, Toronto) and titered on HeLa cells as previously described.48

The ecotropic oncoretroviral packaging cell line E86/pMFG/α-gal A/IRES/huCD25 clone 21 (RV/α-gal A/huCD25) was constructed to produce virus engineered to express both α-gal A and huCD25 as previously described.13 As a control, E86/pUMFG/enYFP (RV/enYFP) was used, which has the same vector backbone and expresses enYFP.49 Cells (4×106) were seeded in 15-cm dishes, and medium containing virus was harvested after 72 hours. Viral titer was determined by infection of 3T3 cells.

Infected cells were then analyzed 72 hours later by flow cytometry to detect either huCD25 or enYFP. huCD25 expression was detected using a phycoerythrin-conjugated antibody against CD25 (α-CD25-phycoerythrin; BD Bioscience Canada, Mississauga, Toronto) and enYFP expression was measured directly. Flow cytometry was performed using the FACSCalibur and analyzed using the CELLQuest software (BD Bioscience Canada).

Establishment of huCD25-Expressing Murine Leukemia Cell Line.

C1498 cells were infected with LV/α-gal A/huCD25 at a multiplicity of infection of 10 productively infectious particles per cell. Cells were re-suspended in filtered viral supernatant supplemented with 8 μg/ml protamine sulfate and overlaid onto plates coated with fibronectin (Roche). Infected C1498 cells were sorted by magnetic activated cell sorting into pools and by flow cytometry on the basis of expression of huCD25 into single-cell clones (C1498/huCD25).

### In Vitro Clearance of Retrovirally Transduced Cells.

Transduced C1498 cell pools, C1498/huCD25 or C1498 NT, were plated in triplicate at a density of 1×104 cells/well in a 96-well plate in a volume of 100 μl. C1498/huCD25 cells were incubated with increasing concentrations (0.1-10 nM) of either of the following reagents: AT antibody, ATS, control IgG-SAP,

or SAP (Advanced Targeting Systems, San Diego, Calif.). C1498 NT cells were treated with ATS at the same concentrations. Cells were incubated at 37° C. and growth inhibition and cell death were then assessed. All treatments were tested in at least two independent experiments.

To assess growth inhibition, 10 μl of 5 mg/ml MTT labeling reagent (Sigma) was added to each well 72 hours after seeding cells. Plates were incubated for 4 hours at 37° C. in a humidified incubator with 5% CO2. Then 100 μl of solubilizing solution (10% sodium dodecyl sulfate, 0.01 M HCl) was added and plates were incubated at 37° C. overnight.

Cell death was assessed 48 hours after seeding by the measurement of lactate dehydrogenase release using the CytoTox 96 Non-Radioactive Cytotoxicity Assay Kit (Promega, Madison, Wis.) as per the manufacturer's instructions.

### Establishment of In Vivo Leukemia Model.

C1498/huCD25 cells were used to generate a leukemia model in Fabry mice.50 Mice were lethally irradiated (11 Gy), and 4 hours later 1×106 C1498/huCD25 cells were injected into the tail vein along with 1×106 fresh BMMNCs isolated by flushing the femurs and tibias of syngeneic donor Fabry mice. Control mice were injected with 1×106 C1498 NT cells. All recipient mice were treated with 5 μg ATS or equimolar (24.4 pmol) amounts of either AT or IgG-SAP on days 2, 4, and 6 after cell transplantation, by injection into the intraperitoneal cavity in a volume of 200 μl. Mice were monitored daily for evidence of disease or distress in compliance with standards set by the Animal Care Committee of the University Health Network.

### In Vivo Clearance of Gene-Corrected Cells in a BMT Model.

Donor Fabry mice were treated with 150 mg/kg 5-fluorouracil (Sigma). Three days later, BM was isolated by flushing the femurs and tibias of treated donor Fabry mice. Mononuclear cells were isolated by centrifugation on Nycoprep and stimulated for 12 hours in Dulbecco's modified Eagle's medium supplemented with 10% fetal calf serum, 2 mM I-glutamine, 1 mM sodium pyruvate, 100 U/ml penicillin, 100 mg/ml streptomycin, 50 ng/ml stem cell factor, 20 ng/ml Flt3 ligand, and 20 ng/ml IL-6. All cytokines were obtained from R&D Systems (Minneapolis, Minn.). Cells were transduced twice (at 12-hour intervals) using supernatant from RV/α-gal A/huCD25 or RV/enYFP producer cell lines13 at multiplicities of infection of approximately 3 and 1 infectious particles per cell, respectively. Infections were performed on plates coated with fibronectin (Roche) and viral supernatant was supplemented with the same cytokine cocktail as above plus 8 μg/ml protamine sulfate (Sigma).

Recipient Fabry mice were lethally irradiated (11 Gy), and 4 hours later infected cells were injected via the tail vein. Cell doses were 0.4×106 and 0.3×106 cells/mouse for the groups transplanted with cells infected with RV/α-gal A/huCD25 and RV/enYFP, respectively. From 4 weeks after transplantation, PB cells were monitored every 4 weeks to detect engraftment. Eight weeks after transplantation, mice were treated intraperitoneally with three doses of 5 μg ATS or equimolar amounts of either AT or IgG-SAP. Doses were administered every 2 days. At 10 weeks after transplantation, PB was analyzed for response to the immunotoxins.

A fourth dose of immunotoxin was administered, as before,11 weeks after transplantation and the animals were killed 12 weeks after transplantation.

Soluble Human CD25 ELISA.

Plasma was isolated from PB of mice by centrifugation at 16,000 for 20 minutes. The level of sCD25 was measured by a direct ELISA using the BD OptEIA Human IL-2 sRα ELISA Set (BD Bioscience Canada) as per the manufacturer's instructions. Each sample was measured in triplicate. α-gal A activity assay. α-gal A activity was measured by a microtiter plate-based fluorometric assay using 5 mM 4-methylumbelliferyl α-dgalactopyranoside (Research Products International, Mount Prospect, Ill.) as the substrate for α-gal A, and 0.1 M N-acetyl-d-galactosamine (Sigma) as an inhibitor of α-N-acetylgalactosaminidase, as previously described.9 Plasma was added directly to the plate in triplicate repeats for each analysis.

For measurement of organ enzyme activity, frozen tissue samples were homogenized and lysates prepared as previously described.12 Plates were read on a fluorescence microtiter plate reader (Dynex, Chantilly, Va.) against nine independent dilutions of a 4-methylumbelliferone standard (Sigma). The protein concentrations of tissue samples were determined using the BCA Protein Assay Kit (Pierce, Rockford, Ill.).

### Statistical Analysis.

Data presented represent means of triplicate determinations for each sample and are representative of results obtained from independent experiments that produced similar relative results.

Differences between groups for enzyme assays and ELISAs were assessed using Student's t-test. The Kaplan-Meier product-limit method was

used to assess the survival of mice and the log-rank statistic was used to test differences between groups (Excel, Microsoft Corporation). Values of P<0.05 were considered to be statistically significant.

### Results

In Vitro Effect of Targeting huCD25 with a Specific Immunotoxin.

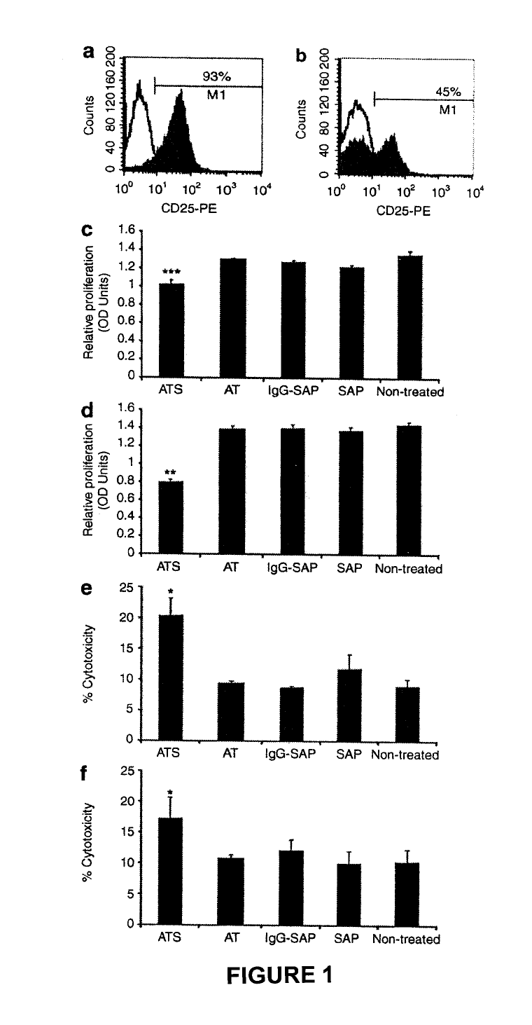

The inventors first determined the specificity and efficacy of the huCD25-targeted immunotoxin ATS. A murine myeloid leukemia cell line, C1498, was infected with a lentiviral vector pHR′cPPTEF-α-gal A-IRES-huCD25-W-SIN (LV/α-gal A/huCD25) that is engineered to express both human α-gal A and huCD25.12 Infected pools were enriched for expression of huCD25 by magnetic activated cell sorting. Two populations of cells were tested that have a broad spectrum of huCD25 expression with a 5 nM concentration of each reagent: ATS, AT, control immunogloblin (Ig)G Ab conjugated to SAP (IgG-SAP), or SAP only. These populations, shown in FIGS. 1a and b, were 90 and 45% positive for huCD25 expression, respectively. MTT [3-(4,5-dimethyl-2-thiazolyl)-2,5-diphenyl-2H-tetrazolium bromide] assays confirmed that both populations of cells treated with ATS showed reduced proliferation (FIGS. 1c and d) and increased cell death as measured by lactate dehydrogenase release (FIGS. 1e and f) compared with cells treated with other reagents. Non-transduced cells did not show any inhibition of proliferation or increased cytotoxicity when treated with ATS.

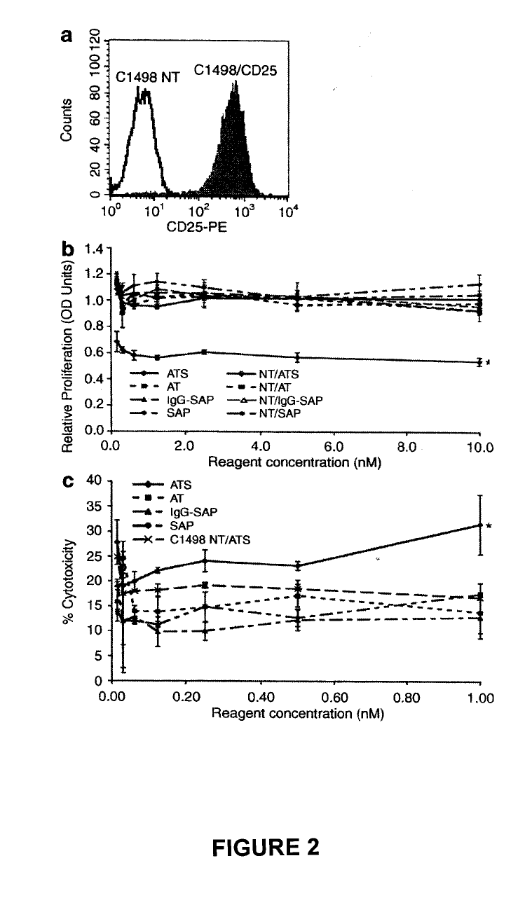

Next, the inventors tested the ability of ATS to clear a clonal population of transduced cells. A single-cell clone expressing huCD25 (C1498/huCD25) was isolated from the infected pool of cells by flow cytometry-based sorting (FIG. 2a). Both C1498/huCD25 and C1498 non-transduced (C1498 NT) cells were incubated with increasing concentrations of each reagent. The effects on cellular proliferation and cell killing were then measured. As shown in FIG. 2b, inhibition of cellular proliferation was significantly higher (P<0.001) when cells were treated with ATS than when cells were treated with control reagents. This effect was specific to cells expressing huCD25, as C1498 NT cells treated with ATS did not show the same level of impaired growth. Similar results were obtained from a lactate dehydrogenase assay. At low doses (<1 nM), cell killing was higher in cells treated with ATS than in cells treated with control reagents (P<0.001) (FIG. 2c).

Clearance of huCD25-Expressing Cells In Vivo Leukemia Model.

As a first step toward determining whether treatment with a CD25 antibody or immunotoxin could clear huCD25-expressing leukemic cells in our mouse model of Fabry disease, the dose of C1498 leukemia cells to use in this strain was optimized. Increasing doses (1×10³ to 1×10⁶) of C1498 NT cells were injected into Fabry mice and the effects were monitored.

Although leukemic cells were not present in the peripheral blood, systemic subcutaneous invasion were present, splenomegaly, and lymphoadenopathy, which mimics some leukemic phenotypes.

For cell doses of 1×10³ and 1×10⁴ cells/mouse, it was found that 100 and 70% of mice, respectively, survived the challenge. For higher cell doses of 1×10⁵ and 1×10⁶ cells/mouse, 100% of the mice succumbed to the leukemia within 60 days and 30 days, respectively. To obtain a more clinically relevant leukemia model, a cell dose of 1×10⁶ cells/mouse for future studies was chosen as at this higher cell dose the phenotype of the transplanted mice progressed to the disease state more quickly and aggressively.

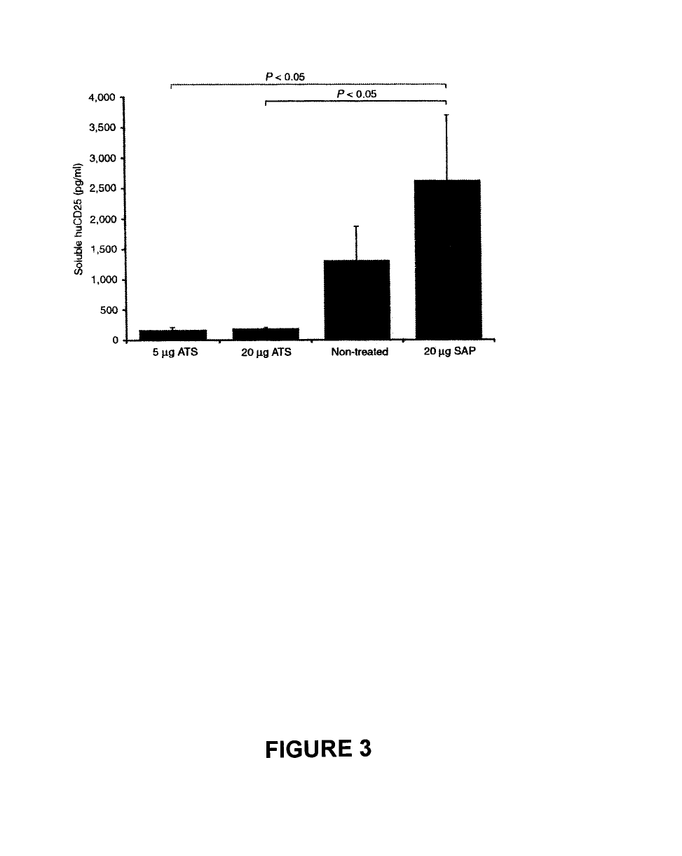

As no previous in vivo studies have been carried out with murine ATS and most studies using other AT derivatives use receptor-saturating doses of antibody,22 it was next necessary to determine an effective dose of immunotoxin. The ability of two different doses of ATS to eliminate huCD25-expressing cells in Fabry mice challenged with C1498/huCD25 leukemia was tested. Mice were lethally irradiated and injected with 1×10⁶ C1498/huCD25 cells and supportive syngeneic BM cells. At days 2, 4, and 6 after leukemic transplant, animals were injected with either 5 μg ATS or 20 μg ATS, injected with SAP only, or left untreated (n=3 per group). Eleven days after challenge, blood was sampled and plasma analyzed for levels of sCD25 by enzyme-linked immunosorbent assay (ELISA). Evaluation of sCD25 levels is a common method used in the clinical setting to monitor tumor burden and treatment response in patients with CD25-expressing lymphoma and leukemia.23 This method also allows sensitive detection of the presence of CD25-positive cells for such studies as it can reflect contributions from abstruse populations. As shown in FIG. 3, treatment with ATS significantly reduced sCD25 (P<0.05) levels compared with animals treated with the control reagent, SAP, and with those left untreated. As treatment with the lower dose of 5 μg of ATS had a similar effect to treatment with the 20 μg dose (FIG. 3), the lower dose of ATS was chose for use in future experiments because this was more cost-effective and may lower the risk of secondary or non-specific toxicities.

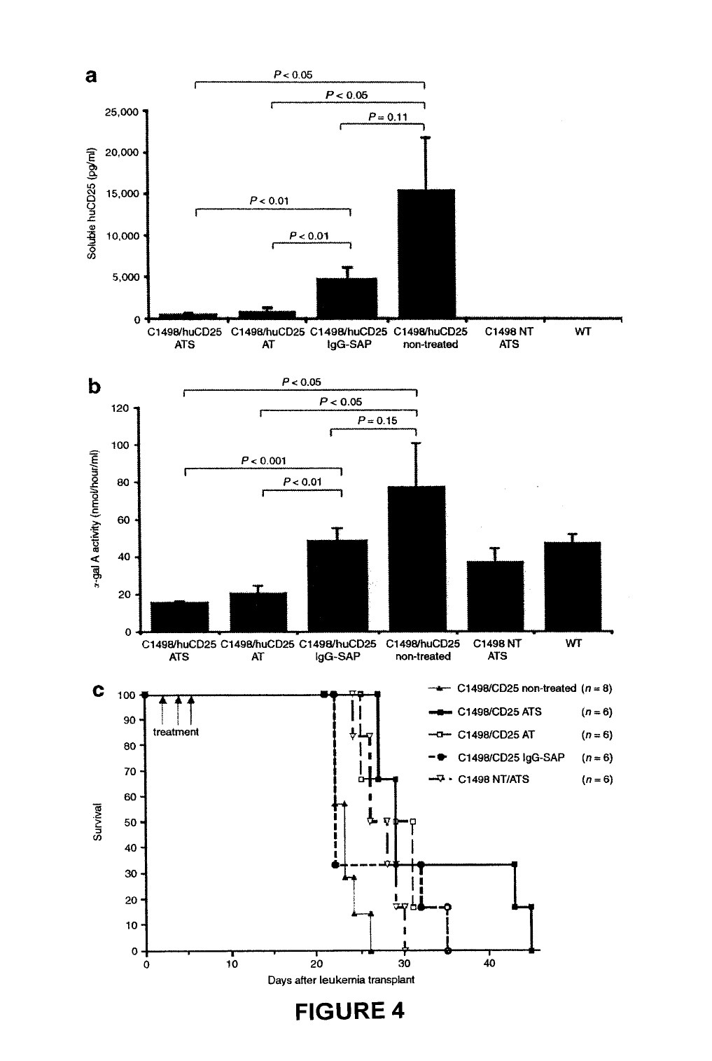

To test the efficacy of our CD25-targeting approach further, a larger experiment using 5 μg ATS was then performed. Mice were lethally irradiated and injected with 1×10⁶ C1498/huCD25 or C1498 NT cells along with supportive syngeneic BM cells via the tail vein. Mice transplanted with C1498/huCD25 cells were then treated with equimolar amounts of ATS, AT, or IgG-SAP. Mice transplanted with C1498 NT cells were treated with 5 μg ATS as a control. All animals were bled on days 7, 11, and 18 after transplantation, and levels of sCD25 in the plasma were measured by ELISA. As shown in FIG. 4a, in mice challenged with C1498/huCD25 cells, at 18 days after transplantation average plasma sCD25 levels were significantly lower in animals treated with ATS (474 pg/ml) and AT (848 pg/ml) than in mice treated with IgG-SAP (4,762 pg/ml; P<0.01) or not treated (15,450 pg/ml; P<0.05). This indicates a lower tumor burden in mice treated with both CD25-targeted reagents, ATS and AT.

The inherent α-gal A deficiency of Fabry mice and the fact that the transplanted tumor cells were engineered to express α-gal A meant that differences in α-gal A activity itself could be used as another surrogate marker of tumor burden. Therefore, plasma α-gal A activity was measured and was found to be lowest in mice treated with ATS (16 nmol/hour/ml) and AT (21 nmol/hour/ml) (FIG. 4b). These levels were significantly lower than those in mice that received IgG-SAP (47 nmol/hour/ml; P<0.001 and P<0.01, versus ATS and AT, respectively) or that were left untreated (77 nmol/hour/ml; P<0.05). Therefore, both ATS and AT are able to de-bulk tumor burden in this huCD25-expressing leukemia model.

To determine the ability of anti-CD25 antibodies to affect survival, animals were monitored daily; a Kaplan-Meier representation of survival is shown in FIG. 4c. In mice treated with ATS, the median survival duration was 29 days. This was significantly higher (P<0.01) than that seen in mice that were not treated (median survival=23 days). Increased survival was also seen in mice treated with AT (median survival of 30 days, P<0.05 versus untreated mice). Therefore, even in the context of a very high leukemic burden, treatment with CD25-targeted antibodies increased survival compared with control treatments. Note that these results are representative of two independent experiments.

### BMT Model

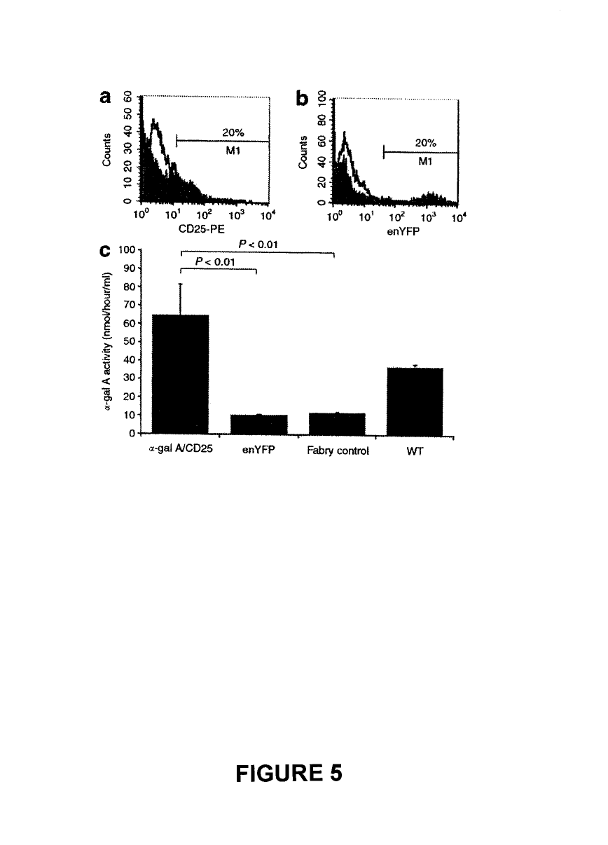

The clearance strategy in the context of a therapeutic BMT model was next tested. BMT is a common gene therapy approach,24 and incorporation of a cell surface protein that can be targeted can improve the safety of the system. Murine bonemarrow mononuclear cells (BMMNCs) were isolated and infected twice with one of two ecotropic oncoretroviral vectors, either E86/pMFG/α-gal A/IRES/huCD25 clone 21 (RV/α-gal A/huCD25) orE86/pUMFG/enYFP (RV/enYFP).13 Flow cytometry analysis of these transduced BMMNCs showed that cells infected with RV/α-gal A/huCD25 were approximately 30% positive for expression of huCD25 (FIG. 5a) and cells infected with RV/enYFP were approximately 20% positive for enhanced yellow fluorescent protein(enYFP) expression (FIG. 5b). Cells were then injected into lethally irradiated Fabry mice, which were monitored monthly for engraftment.

At 8 weeks after transplantation, plasma from recipient Fabry mice was analyzed for α-gal A activity and for levels of sCD25. Average plasma α-gal A activity in mice transplanted with BMMNCs infected with RV/α-gal A/huCD25 was 65 nmol/hour/ml, approximately sixfold higher than in both control Fabry mice and mice transplanted with RV/enYFP-infected BMMNCs (FIG. 5c). This indicates that a therapeutic correction twofold higher than in normal C57BL/6 mice was achieved (FIG. 5c). At this time, the average level of sCD25 in the plasma of Fabry mice transplanted with BMMNCs infected with RV/α-gal A/huCD25 was 1212±370 pg/ml. In contrast, sCD25 was undetectable in mice transplanted with RV/enYFP-infected cells, in wild-type C57BL/6 mice, and in untreated Fabry mice.

Mice were then treated with either ATS, AT, or IgG-SAP, as in our leukemia model (see above). Seven days after the third dose of immunotoxin, plasma was sampled to determine the effect of treatment. Comparisons were made with pre-treatment values collected for each mouse at 8 weeks after transplantation.

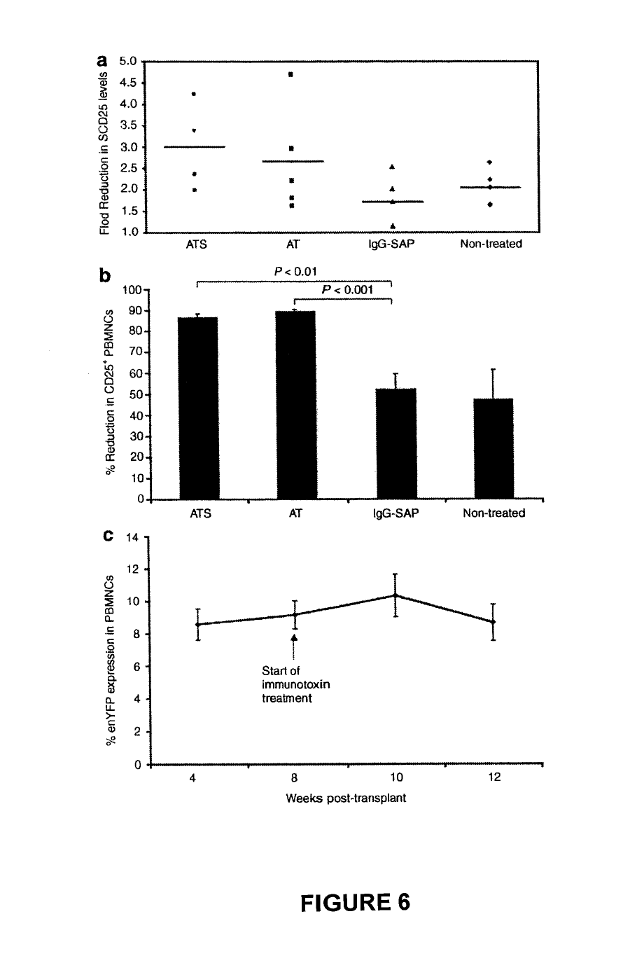

As shown in FIG. 6a, treatment with ATS resulted in lower plasma sCD25 levels than in mice that were treated with IgG-SAP or mice that were not treated (P<0.05). In addition, analysis of huCD25 expression on peripheral blood (PB) mononuclear cells by flow cytometry showed that mice treated with ATS had significantly reduced numbers of huCD25-expressing PB mononuclear cells than mice treated with IgG-SAP (P<0.01) or untreated mice (P<0.05) (FIG. 6b). Similar effects were observed in mice treated with AT, further supporting the conceptual ability of targeted anti-CD25 antibodies to eliminate retrovirally transduced donor hematopoietic cells in vivo. Expression of enYFP was monitored before and after treatment with ATS and it was found that levels remained stable over the course of the experiment (FIG. 6c), demonstrating the specificity of the immunotoxin for cells expressing huCD25.

To examine the effect of a later administration of antibody or immunotoxin, one final dose was administered and then mice were killed. Enzyme activity was measured in various tissues to determine the systemic effect of each reagent. PB mononuclear cells from mice that were treated with ATS showed significantly lower (P<0.05) α-gal A activity than mice treated with IgG-SAP

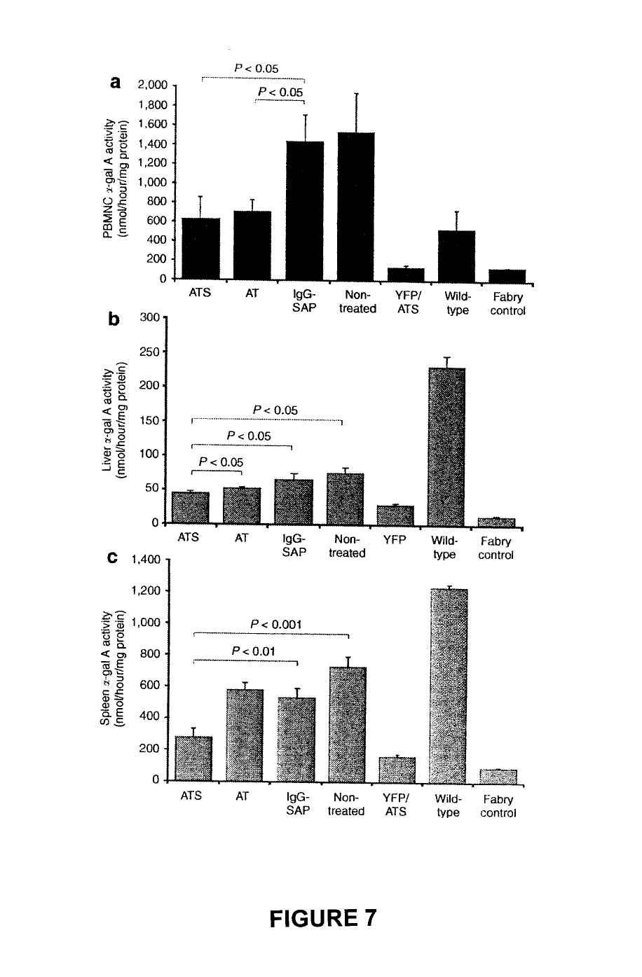

(FIG. 7a). Similarly, α-gal A activity in the livers of mice treated with ATS was significantly lower (P<0.05) than enzyme activity in the livers of mice treated with AT or IgG-SAP or untreated mice (FIG. 7b). Likewise, in the spleens of mice treated with ATS, there was significantly lower (P<0.01) α-gal A activity than in IgG-SAP-treated or untreated mice (FIG. 7c).

### Discussion

Gene therapy is the most promising curative treatment for monogenetic diseases such as lysosomal storage disorders.25 Although considerable advances toward the development of retrovirus-based gene therapy strategies for Fabry disease have been made, concerns remain regarding the safety of integrating vectors.

To address this issue, a cell surface marker such as huCD25 acts as an effective built-in safety mechanism in the event of insertional genotoxicity by facilitating the clearance of transduced cells with a specifically targeted immunotoxin. huCD25 in combination with α-galA in studies evaluating the efficacy of retroviral gene therapy for Fabry disease has been previously described.12,13 No untoward effects of exogenously expressing this protein were observed nor have altered therapeutic effects of this surface antigen on α-gal A-mediated correction in vivo been seen.

Monoclonal antibodies have been successfully used in the clinic for many years to treat hematological malignancies, with minimal toxicity.26,27 For instance, rituximab, an anti-CD20 antibody, has been used to treat a variety of lymphoid malignancies.26,28-30 In addition, a strategy for clearing transduced hematopoietic cells in vivo using an anti-CD20 Ab was proposed for the treatment of graft-versus-host disease.31 The premise is that T cells can be transduced with a viral vector carrying the complementary DNA for CD20 before BMT, and if graft-versus-host disease occurs, then anti-CD20 antibodies can be used to eliminate the donor T cells. These studies have shown promising results in vitro; however, no studies have been carried out to demonstrate efficacy in vivo.32,33.

Aberrant levels of CD25 expression characterize numerous disorders such as adult T-cell leukemia/lymphoma, Hodgkin's lymphoma, hairy cell leukemias, and true histiocytic lymphomas.34 Treatment of these diseases using antibodies against CD25, as well as newer recombinant immunotoxins, has resulted in complete and partial remissions in patients.34,35 Currently, anti-CD25 antibodies are widely used for the prevention of renal graft rejection and in some cases for prophylactic treatment against graftversus-host disease.36,37 Furthermore, studies have shown that when anti-CD25 antibodies are used to deplete CD4+CD25+ regulatory T cells, anti-tumor immunity is enhanced.38-40 These findings provided the rationale for using anti-CD25 toxinconjugated antibodies to target huCD25.

The inventors have shown both in vitro in cell culture and in vivo in a Fabry mouse model, that a CD25-targeted treatment can specifically and effectively kill leukemia cells that express both a therapeutic transgene, α-gal A, and huCD25 following infection with a retroviral vector. In a model using huCD25-expressing C1498 leukemia cells, measurement of sCD25 levels and α-gal A activity following ATS treatment showed a 32- and 5-fold reduction over untreated mice, respectively. Similar results were obtained when mice were treated with AT. In addition, treatment with either ATS or AT extended survival by approximately 26% over mice that were not treated. It was not unexpected that despite this increase in survival time, these mice still succumbed to the leukemia, as a very high tumor dose was chosen for administration. As has been observed in clinical trials for the treatment of naturally occurring CD25-expressing leukemias, better outcomes are achievable with multi-modal therapy.41-43 These results demonstrate proof of principle that the clearance strategy can de-bulk tumor burden and extend survival.

The clearance strategy is useful as a safety mechanism against retroviral-induced genotoxicity in hematopoietic stem/progenitor cells. The system was next examined in a BMT setting in a mouse model of Fabry disease using an oncoretroviral vector gene delivery vehicle.13 An oncoretroviral vector was chosen here. Oncoretroviral vectors have a greater propensity for integrating near transcriptional start sites, proto-oncogenes, and cell cycle regulatory genes than do lentiviral vectors,44-46 perhaps making them more likely to cause dysregulation in gene expression leading to leukemias,4 for example. After our standard gene transfer and BMT protocol in Fabry mice, supra-physiological levels of α-gal A activity in the plasma of transplanted mice was achieved. Anti-CD25-targeted treatment of transplanted mice decreased levels of α-gal A activity in PB mononuclear cells and decreased expression of huCD25 on these cells, indicating clearance of the transduced cell population itself. As expected, a corresponding decrease in the level of sCD25 in the PB was seen. This corresponds well with data showing a positive correlation between levels of sCD25 and α-gal A activity in the PB of mice treated with LV/α-gal A/huCD25.12

ATS treatment was more effective at clearing transduced cells from the organs than AT. ATS treatment resulted in a systemic decrease in organ α-gal A activity, indicating that there was widespread elimination of transduced cells. The study further provides evidence that ATS is not merely clearing circulating sCD25 directly but is targeting and killing the CD25-expressing cells.

This is the first report of an antibody-mediated clearance strategy being applied to gene therapy in the context of a therapeutic BMT. The complementary DNA for CD25 was recently cloned from the rhesus macaque, which will facilitate this endeavor.47 A variety of cell surface proteins are readily incorporated into various retroviral vectors in combination with any therapeutic transgene. Using this system will add another safety mechanism to retroviral gene transfer systems.

### Example 2

### CD19 Immunotoxin Experiments

The efficacy of a CD19 monoclonal antibody conjugated to an immunotoxin to specifically clear cells transduced with pCCL.SIN.cPPT.EF.CD19ΔTmpkF105YR200A.WPRE will be tested in vitro and in vivo.

### In Vitro

Jurkat cells will be transduced with pCCL.SIN.cPPT.EF.CD19ΔTmpkF105YR200A.WPRE. The transduced pool of cells will be enriched for cells expressing CD19 using fluorescence-activated cell sorting (FACS). The CD19 enriched population and a non-transduced control group will be cultured in the presence of various concentrations of CD19 immunotoxin for 2-4 days. Cell proliferation will then be assessed using the Cell Titer 96 Aqueous One Solution Cell Proliferation Assay Kit (Promega) and cell death will be measured using the CytoTox 96 Cytotoxicity Assay Kit (Promega). It is expected that transduced cells cultured with CD19 immunotoxin will show a significant decrease in proliferation and a significant increase in cytotoxicity compared to control groups. To model a potential clinical adverse advent in which a patient might develop a malignancy such as leukemia, these experiments will also be performed using K562 erythroid leukemia cells transduced with pCCL.SIN.cPPT.EF.CD19ΔTmpkF105YR200A.WPRE.

### In Vivo

Murine bone marrow cells will be isolated and transduced ex vivo with pCCL.SIN.cPPT.EF.CD19ΔTmpkF105YR200A.WPRE. Transduced cells and a non-transduced control group will then be injected into irradiated non-obese diabetic/severe combined immunodeficiency (SCID) mice. Approximately 8 weeks post transplant, the levels of CD19 in the peripheral blood will be measured using flow cytometry. Transplanted mice will then be given an intraperitoneal injection of CD19 immunotoxin or placebo as a control every 2 days for a total of three injections. Clearance of CD19-positive cells in the peripheral blood will be measured using flow cytometry. It is expected that mice transplanted with pCCL.SIN.cPPT.EF.CD19ΔTmpkF105YR200A.WPRE transduced cells and treated with CD19 immunotoxin will show significantly decreased levels of CD19 expressing cells in the peripheral blood compared to control groups. Again, to model a potential clinical adverse event such as leukemia, a second experiment in which K562 erythroid leukemia cells are used will be done. K562 cells will be transduced with pCCL.SIN.cPPT.EF.CD19ΔTmpkF105YR200A.WPRE and enriched for CD19 expression using FACS. The enriched K562 population and a non-transduced control group will be injected into the flank of NOD/SCID mice. After transplantation, mice will be given IP injections of CD19 immunotoxin or a placebo as a control every 2 days for 3 a total of 3 injections. The growth of the tumors will be monitored. It is expected that mice transplanted with K562 cells transduced with pCCL.SIN.cPPT.EF.CD19ΔTmpkF105YR200A.WPRE and treated with CD19 immunotoxin will show significantly decreased rates of tumor growth compared to control groups.

### Example 3

### CD19 Immunotoxin Experiments

The efficacy of a monoclonal antibody against CD19 conjugated to an immunotoxin (CD19-IT) to specifically clear cells transduced with pCCL.SIN.cPPT.EF.CD19ΔTmpkF105YR200A.WPRE. will be tested in vitro and in vivo.

### In Vitro

Jurkat cells will be transduced with pCCL.SIN.cPPT.EF.CD19ΔTmpkF105YR200A.WPRE. The transduced pool of cells will be enriched for cells expressing CD19 using fluorescence-activated cell sorting (FACS). The CD19 enriched population and a non-transduced control group will be cultured in the presence of various concentrations of CD19-IT for 2-4 days. Cell proliferation will then be assessed using the Cell Titer 96 Aqueous One Solution Cell Proliferation Assay Kit (Promega) and cell death will be measured using the CytoTox 96 Cytotoxicity Assay Kit (Promega). It is expected that transduced cells cultured with CD19-IT will show a significant decrease in proliferation and a significant increase in cytotoxicity compared to control groups. CD19-IT will also be tested in combination with AZT to determine if transduced cells that are not sensitive to AZT can subsequently be eliminated by administration of CD19-IT. The CD19 enriched Jurkat population and a non-transduced control group will be cultured in 100 μM AZT for 4 days with media and AZT being replaced each day. Cell proliferation and cell death will be measured as described above. Cells will then be cultured in the presence of CD19-IT for 2-4 days and proliferation and cell death will be measured again. It is expected that approximately 80% of transduced cells will be killed by treatment with AZT, and that subsequent culture with CD19-IT will cause a further significant decrease in viable cell number compared to control groups. To model a potential clinical adverse advent in which a patient might develop a malignancy such as leukemia, these experiments will also be performed using K562 erythroid leukemia cells transduced with pCCL.SIN.cPPT.EF.CD19ΔTmpkF105YR200A.WPRE to show cell killing.

### In Vivo

Murine bone marrow cells will be isolated and transduced ex vivo with pCCL.SIN.cPPT.EF.CD19ΔTmpkF105YR200A.WPRE. Transduced cells and a non-transduced control group will then be injected into irradiated non-obese diabetic/severe combined immunodeficiency (NOD/SCID) mice. Approximately 8 weeks post transplant, the levels of CD19 expressing cells in the peripheral blood will be measured using flow cytometry. Transplanted mice will then be given an intraperitoneal (IP) injection of CD19-IT or placebo as a control every 2 days for a total of three injections. Clearance of CD19-positive cells in the peripheral blood will be measured using flow cytometry. It is expected that mice transplanted with pCCL.SIN.cPPT.EF.CD19ΔTmpkF105YR200A.WPRE transduced cells and treated with CD19-IT will show significantly decreased levels of CD19 expressing cells in the peripheral blood compared to control groups. Another group to be included in this experiment will examine if treatment with AZT in combination with CD19-IT can further reduce levels of CD19 expressing cells in the peripheral blood. In this group, mice will receive IP injections of AZT everyday for 14 days. At this point, CD19 levels in the peripheral blood will be measured using flow cytometry. Mice will then be administered IP injections of CD19-IT every 2 days for a total of 3 injections. CD19 levels in the peripheral blood will then be measured again. It is expected that this combination therapy will reduce levels of CD19 expressing cells in the peripheral blood compared to groups treated with either AZT or CD19-IT alone, and to control groups.

Again, to model a potential clinical adverse event such as leukemia, a second experiment in which K562 erythroid leukemia cells are used will be performed. K562 cells will be transduced with pCCL.SIN.cPPT.EF.CD19ΔTmpkF105YR200A.WPRE and enriched for CD19 expression using FACS. The enriched K562 population and a non-transduced control group will be injected into the flank of NOD/SCID mice. After transplantation, mice will be given IP injections of CD19-IT or a placebo as a control every 2 days for 3 a total of 3 injections. The growth of the tumors will be monitored. It is expected that mice transplanted with K562 cells transduced with pCCL.SIN.cPPT.EF.CD19ΔTmpkF105YR200A.WPRE and treated with CD19-IT will show significantly decreased rates of tumor growth compared to control groups. Treatment with a combination of AZT and CD19-IT is also tested in a method similar to that described above and provides cell killing.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Aiuti, A, Slavin, S, Aker, M, Ficara, F, Deola, S, Mortellaro, A et al. (2002). Correction of ADA-SCID by stem cell gene therapy combined with nonmyeloablative conditioning. Science 296: 2410-2413.
2. Gaspar, H B, Parsley, K L, Howe, S, King, D, Gilmour, K C, Sinclair, J et al. (2004). Gene therapy of X-linked severe combined immunodeficiency by use of a pseudotyped gammaretroviral vector. Lancet 364: 2181-2187.
3. Robbins, P D and Ghivizzani, S C (1998). Viral vectors for gene therapy. Pharmacol Ther 80: 35-47.
4. Hacein-Bey-Abina, S, Von Kalle, C, Schmidt, M, McCormack, M P, Wulffraat, N, Leboulch, P et al. (2003). LMO2-associated clonal T cell proliferation in two patients after gene therapy for SCID-X1. Science 302: 415-419.
5. McCormack, M P and Rabbitts, T H (2004). Activation of the T-cell oncogene LMO2 after gene therapy for X-linked severe combined immunodeficiency. N Engl J Med 350: 913-922.
6. Baum, C, von Kalle, C, Staal, F J, Li, Z, Fehse, B, Schmidt, M et al. (2004). Chance or necessity? Insertional mutagenesis in gene therapy and its consequences. Mol Ther 9: 5-13.
7. Brady, R O, Gal, A E, Bradley, R M, Martensson, E, Warshaw, A L and Laster, L (1967). Enzymatic defect in Fabry's disease. Ceramidetrihexosidase deficiency. N Engl J Med 276: 1163-1167.
8. Kase, R, Shimmoto, M, Itoh, K, Utsumi, K, Kotani, M, Taya, C et al. (1998). Immunohistochemical characterization of transgenic mice highly expressing human lysosomal alpha-galactosidase. Biochim Biophys Acta 1406: 260-266.
9. Medin, J A, Tudor, M, Simovitch, R, Quirk, J M, Jacobson, S, Murray, G J et al. (1996). Correction in trans for Fabry disease: expression, secretion and uptake of alpha-galactosidase A in patient-derived cells driven by a high-titer recombinant retroviral vector. Proc Natl Acad Sci USA 93: 7917-7922.
10. Takenaka, T, Murray, G J, Qin, G, Quirk, J M, Ohshima, T, Qasba, P et al. (2000). Long-term enzyme correction and lipid reduction in multiple organs of primary and secondary transplanted Fabry mice receiving transduced bone marrow cells. Proc Natl Acad Sci USA 97: 7515-7520.
11. Yoshimitsu, M, Higuchi, K, Ramsubir, S, Nonaka, T, Rasaiah, V I, Siatskas, C et al. (2007). Efficient correction of Fabry mice and patient cells mediated by lentiviral transduction of hematopoietic stem/progenitor cells. Gene Ther 14: 256-265.
12. Yoshimitsu, M, Sato, T, Tao, K, Walia, J S, Rasaiah, V I, Sleep, G T et al. (2004). Bioluminescent imaging of a marking transgene and correction of Fabry mice by neonatal injection of recombinant lentiviral vectors. Proc Natl Acad Sci USA 101: 16909-16914.
13. Qin, G, Takenaka, T, Telsch, K, Kelley, L, Howard, T, Levade, T et al. (2001). Preselective gene therapy for Fabry disease. Proc Natl Acad Sci USA 98: 3428-3433.
14. Taniguchi, T and Minami, Y (1993). The IL-2/IL-2 receptor system: a current overview. Cell 73: 5-8.
15. Gaffen, S L (2001). Signaling domains of the interleukin 2 receptor. Cytokine 14: 63-77.
16. Hanisch, U K and Quirion, R (1995). Interleukin-2 as a neuroregulatory cytokine. Brain Res Brain Res Rev 21: 246-284.
17. David, D, Bani, L, Moreau, J L, Demaison, C, Sun, K, Salvucci, O et al. (1998). Further analysis of interleukin-2 receptor subunit expression on the different human peripheral blood mononuclear cell subsets. Blood 91: 165-172.
18. Rubin, L A, Kurman, C C, Fritz, M E, Biddison, W E, Boutin, B, Yarchoan, R et al. (1985). Soluble interleukin 2 receptors are released from activated human lymphoid cells in vitro. J Immunol 135: 3172-3177.
19. Uchiyama, T, Broder, S and Waldmann, T A (1981). A monoclonal antibody (anti-Tac) reactive with activated and functionally mature human T cells. I. Production of anti-Tac monoclonal antibody and distribution of Tac (+) cells. J Immunol 126: 1393-1397.
20. Lappi, D A, Esch, F S, Barbieri, L, Stirpe, F and Soria, M (1985). Characterization of a Saponaria officinalis seed ribosome-inactivating protein: immunoreactivity and sequence homologies. Biochem Biophys Res Commun 129: 934-942.
21. Barbieri, L, Valbonesi, P, Bonora, E, Gorini, P, Bolognesi, A and Stirpe, F (1997). Polynucleotide:adenosine glycosidase activity of ribosome-inactivating proteins: effect on DNA, RNA and poly(A). Nucleic Acids Res 25: 518-522.
22. Zhang, Z, Zhang, M, Garmestani, K, Talanov, V S, Plascjak, P S, Beck, B et al. (2006). Effective treatment of a murine model of adult T-cell leukemia using 211At-7G7/B6 and its combination with unmodified anti-Tac (daclizumab) directed toward CD25. Blood 108: 1007-1012.
23. Perez-Encinas, M, Villamayor, M, Campos, A, Gonzalez, S and Bello, J L (1998). Tumor burden and serum level of soluble CD25, CD8, CD23, CD54 and CD44 in non-Hodgkin's lymphoma. Haematologica 83: 752-754.
24. Boelens, J J (2006). Trends in haematopoietic cell transplantation for inborn errors of metabolism. J Inherit Metab Dis 29: 413-420.
25. Futerman, A H and van Meer, G (2004). The cell biology of lysosomal storage disorders. Nat Rev Mol Cell Biol 5: 554-565.
26. Dillman, R O (2001). Monoclonal antibody therapy for lymphoma: an update. Cancer Pract 9: 71-80.
27. Gokbuget, N and Hoelzer, D (2004). Treatment with monoclonal antibodies in acute lymphoblastic leukemia: current knowledge and future prospects. Ann Hematol 83: 201-205.
28. McLaughlin, P, Grillo-Lopez, A J, Link, B K, Levy, R, Czuczman, M S, Williams, M E et al. (1998). Rituximab chimeric anti-CD20 monoclonal antibody therapy for relapsed indolent lymphoma: half of patients respond to a four-dose treatment program. J Clin Oncol 16: 2825-2833.
29. Grillo-Lopez, A J, Hedrick, E, Rashford, M and Benyunes, M (2002). Rituximab: ongoing and future clinical development. Semin Oncol 29: 105-112.
30. Cvetkovic, R S and Perry, C M (2006). Rituximab: a review of its use in non-Hodgkin's lymphoma and chronic lymphocytic leukaemia. Drugs 66: 791-820.
31. Introna, M, Barbui, A M, Bambacioni, F, Casati, C, Gaipa, G, Borleri, G et al. (2000). Genetic modification of human T cells with CD20: a strategy to purify and lyse transduced cells with anti-CD20 antibodies. Hum Gene Ther 11: 611-620.
32. van Meerten, T, Claessen, M J, Hagenbeek, A and Ebeling, S B (2006). The CD20/alphaCD20 ‘suicide’ system: novel vectors with improved safety and expression profiles and efficient elimination of CD20-transgenic T cells. Gene Ther 13: 789-797.
33. Serafini, M, Manganini, M, Borleri, G, Bonamino, M, Imberti, L, Biondi, A et al. (2004). Characterization of CD20-transduced T lymphocytes as an alternative suicide gene therapy approach for the treatment of graft-versus-host disease. Hum Gene Ther 15: 63-76.
34. Kreitman, R J, Wilson, W H, White, J D, Stetler-Stevenson, M, Jaffe, E S, Giardina, S et al. (2000). Phase I trial of recombinant immunotoxin anti-Tac(Fv)-PE38 (LMB-2) in patients with hematologic malignancies. J Clin Oncol 18: 1622-1636.
35. Kreitman, R J, Wilson, W H, Robbins, D, Margulies, I, Stetler-Stevenson, M, Waldmann, T A et al. (1999). Responses in refractory hairy cell leukemia to a recombinant immunotoxin. Blood 94: 3340-3348.
36. Lin, M, Ming, A and Zhao, M (2006). Two-dose basiliximab compared with two-dose daclizumab in renal transplantation: a clinical study. Clin Transplant 20: 325-329.
37. Chen, H R, Ji, S Q, Wang, H X, Yan, H M, Zhu, L, Liu, J et al. (2003). Humanized anti-CD25 monoclonal antibody for prophylaxis of graft-vs-host disease (GvHD) in haploidentical bone marrow transplantation without ex vivo T-cell depletion. Exp Hematol 31: 1019-1025.
38. Fecci, P E, Sweeney, A E, Grossi, P M, Nair, S K, Learn, C A, Mitchell, D A et al. (2006). Systemic anti-CD25 monoclonal antibody administration safely enhances immunity in murine glioma without eliminating regulatory T cells. Clin Cancer Res 12: 4294-4305.
39. Knutson, K L, Dang, Y, Lu, H, Lukas, J, Almand, B, Gad, E et al. (2006). IL-2 immunotoxin therapy modulates tumor-associated regulatory T cells and leads to lasting immune-mediated rejection of breast cancers in neu-transgenic mice. J Immunol 177: 84-91.
40. Onizuka, S, Tawara, I, Shimizu, J, Sakaguchi, S, Fujita, T and Nakayama, E (1999). Tumor rejection by in vivo administration of anti-CD25 (interleukin-2 receptor alpha) monoclonal antibody. Cancer Res 59: 3128-3133.
41. Zhang, M, Zhang, Z, Goldman, C K, Janik, J and Waldmann, T A (2005). Combination therapy for adult T-cell leukemia-xenografted mice: flavopiridol and anti-CD25 monoclonal antibody. Blood 105: 1231-1236.
42. Jaeger, G, Neumeister, P, Brezinschek, R, Hofler, G, Quehenberger, F, Linkesch, W et al. (2002). Rituximab (anti-CD20 monoclonal antibody) as consolidation of first-line CHOP chemotherapy in patients with follicular lymphoma: a phase II study. Eur J Haematol 69: 21-26.
43. Claviez, A, Eckert, C, Seeger, K, Schrauder, A, Schrappe, M, Henze, G et al. (2006). Rituximab plus chemotherapy in children with relapsed or refractory CD20-positive B-cell precursor acute lymphoblastic leukemia. Haematologica 91: 272-273.
44. Montini, E, Cesana, D, Schmidt, M, Sanvito, F, Ponzoni, M, Bartholomae, C et al. (2006). Hematopoietic stem cell gene transfer in a tumor-prone mouse model uncovers low genotoxicity of lentiviral vector integration. Nat Biotechnol 24: 687-696.
45. De Palma, M, Montini, E, Santoni de Sio, F R, Benedicenti, F, Gentile, A, Medico, E et al. (2005). Promoter trapping reveals significant differences in integration site selection between MLV and HIV vectors in primary hematopoietic cells. Blood 105: 2307-2315.
46. Wu, X, Li, Y, Crise, B and Burgess, S M (2003). Transcription start regions in the human genome are favored targets for MLV integration. Science 300: 1749-1751.
47. Silvertown, J D, Walia, J S and Medin, J A (2005). Cloning, sequencing and characterization of lentiviral-mediated expression of rhesus macaque (Macaca mulatta) interleukin-2 receptor alpha cDNA. Dev Comp Immunol 29: 989-1002.
48. Naldini, L, Blomer, U, Gallay, P, Ory, D, Mulligan, R, Gage, F H et al. (1996). In vivo gene delivery and stable transduction of nondividing cells by a lentiviral vector. Science 272: 263-267.

49. Medin, J A, Brandt, J E, Rozler, E, Nelson, M, Bartholomew, A, Li, C et al. (1999). Ex vivo expansion and genetic marking of primitive human and baboon hematopoietic cells. Ann N Y Acad Sci 872: 233-240; discussion 240-242.

50. Ohshima, T, Murray, G J, Swaim, W D, Longenecker, G, Quirk, J M, Cardarelli, C O et al. (1997). alpha-Galactosidase A deficient mice: a model of Fabry disease. Proc Natl Acad Sci USA 94: 2540-2544.

We claim:

1. A suicide gene system comprising:
a. a stably integrating delivery vector;
b. an activator polynucleotide encoding a polypeptide that converts a prodrug to a drug;
c. a docking polynucleotide encoding a cell surface polypeptide that selectively binds a toxic binding agent; and
d. a therapeutic α-galactosidase A polynucleotide;
wherein the suicide gene system induces death in a cell expressing the activator polynucleotide and/or docking polynucleotide when the cell is contacted with the prodrug and/or the toxic binding agent.

2. A composition comprising:
a. a stably integrating delivery vector;
b. an activator polynucleotide encoding a polypeptide that converts a prodrug to a drug;
c. a docking polynucleotide encoding a docking polypeptide that selectively binds an antibody or an antibody conjugated with a toxic agent; and
d. a therapeutic α-galactosidase A polynucleotide.

3. The composition of claim 2, wherein the activator polynucleotide comprises a tmpk polynucleotide with at least 80% sequence identity to a modified tmpk polynucleotide.

4. The composition of claim 2 or 3, wherein the polynucleotide comprises a mammalian polynucleotide, optionally a human polynucleotide, and the polypeptide comprise a mammalian polypeptide, optionally a human polypeptide.

5. The composition any one of claims 2 to 4, wherein the activator polynucleotide comprises a modified mammalian tmpk polynucleotide encoding a modified mammalian tmpk polypeptide that increases phosphorylation of a prodrug relative to phosphorylation of the prodrug by wild-type mammalian tmpk polypeptide, optionally the modified mammalian tmpk polynucleotide comprises a mammalian tmpk polynucleotide with a point mutation or multiple mutations.

6. The composition of claim 5, wherein the point mutation comprises a mutation in a codon of the polynucleotide selected from the group consisting of a mutation that encodes a F to Y mutation at amino acid position 105, a mutation that encodes a R to G point mutation at amino acid position 16, and a mutation that encodes a R to A mutation at amino acid position 200 or combinations of the above.

7. The composition of claim 6, wherein the polynucleotide further comprises all or part of the large lid or small lid domain of E. coli thymidine kinase.

8. The composition of any one of claims 2 to 5, wherein the modified mammalian tmpk polynucleotide has been modified by substituting a portion of wild-type tmpk polynucleotide sequence with an exogenous polynucleotide sequence.

9. The composition of claim 8, wherein the substituted portion comprises all or part of a large lid or small lid domain from E. coli thymidine kinase.

10. The composition of any one of claims 2 to 9, wherein the activator polynucleotide and docking polynucleotide are fused and encode an activator/docking fusion.

11. The composition of any one of claims 2 to 9, further comprising a detection cassette comprising a polynucleotide sequence different than the docking polynucleotide.

12. The composition of anyone of claims 2 to 10, wherein the docking polynucleotide encodes HSA, CD24, CD34, LNGFR, EpoR, CD19, CD25 or CD20, or a fragment thereof that binds an antibody or the toxic binding agent directly.

13. The composition of any one of claims 2 to 12, wherein the toxic binding agent comprises an antibody conjugated to a toxin.

14. The composition of claim 13, wherein the antibody comprises an anti-CD19 antibody, anti-CD20 antibody or anti-CD25 antibody and the toxin comprises saporin.

15. The composition of any one of claims 2 to 13, wherein the delivery vector comprises a retroviral vector, an adenoviral vector, an adeno-associated viral vector, spumaviral vector, a lentiviral vector or a plasmid or other vector described in the application.

16. The composition of claim 15, wherein the delivery vector comprises a lentiviral vector that has a pHR′ backbone and comprises 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR).

17. The composition of claim 15, wherein the delivery vector comprises a lentiviral vector that has a pCCL backbone and comprises 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR).

18. A method of expressing an activator polynucleotide, a docking polynucleotide and a therapeutic α-galactosidase A polynucleotide in a mammalian cell comprising contacting the mammalian cell with the composition of anyone of claims 2 to 17.

19. The method of claim 18, further comprising isolating the cells.

20. The method of any one of claim 18 or 19, wherein the mammalian cell is a an embryonic stem cell, a stem cell, a hematopoietic cell, an iPS cell, a marrow stroma cell, a mesenchymal stem cell, an endothelia progenitor cell, a T cell or a human cell.

21. The method of any one of claims 18 to 20, wherein the mammalian cell is a tumor cell.

22. The method of any one of claims 18 to 21, further comprising a step wherein the isolated mammalian cell is transplanted into a mammal.

23. A method of killing a mammalian cell expressing the activator polynucleotide, a docking polynucleotide and the therapeutic α-galactosidase A polynucleotide of any one of claims 2 to 17, comprising contacting the cell with an effective amount of a prodrug and/or a toxic binding agent to kill the cell.

24. A method of killing a mammalian cell expressing an activator polynucleotide and/or a docking polynucleotide comprising:
a. contacting the mammalian cell with a composition of any one of claims 2 to 17;
b. isolating the cell; and
c. contacting the cell with an effective amount of a prodrug and/or a toxic binding agent to kill the cell.

25. The method of claim 23 or 24, wherein the prodrug is selected from the group consisting of thymidine analog, uracil analog, AZT, dT4 and 5-FU.

26. The method of claim 23 or 24, wherein the toxic binding agent comprises an antibody or an immunotoxin that binds CD19, truncated CD19, CD20, or CD25.

27. An actuable cell destruction component of an expression vector construct comprising:
a. an activator polynucleotide encoding a polypeptide that converts a prodrug to a drug;
b. a docking polynucleotide encoding a cell surface polypeptide that selectively binds a toxic binding agent; and
c. a therapeutic α-galactosidase A polynucleotide.

28. The suicide gene system of claim 1, wherein the activator polynucleotide comprises the activator polynucleotide of any one of claims 2 to 17 and the docking polynucleotide comprises the docking polynucleotide of any one of claims 2 to 17.

29. The suicide system of claim 1 or 28, for use in gene therapy treatment of a subject.

30. The suicide system of claim 1 or 28, for use in cells for transplant into a subject.

31. The suicide system of any one of claim 1, 23 or 28, wherein the cells are killed if the subject develops or is suspected of developing GVHD.

32. A mammalian cell comprising the suicide gene system of any one of claim 1, 23, 28 or 31.

33. A kit comprising the composition of any one of claims 2 to 17, a toxic binding agent such as an immunotoxin and/or a prodrug.

34. A method of medical treatment of Fabry disease in a subject in need thereof, comprising administering to the subject in need thereof the composition of any one of claims 2 to 17 or the cell of claim 32.

35. A method of gene therapy of a subject with Fabry disease, comprising administering to the subject in need thereof the composition of any one of claims 2 to 17.


SEQUENCE LISTING (39 sequences)

SEQ ID NO: 1 - DNA, 639 bp, Homo sapiens
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaagtga

SEQ ID NO: 2 - protein, 212 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 3 - DNA, 639 bp, Homo sapiens; identical to SEQ ID NO: 1

SEQ ID NO: 4 - protein, 212 aa, Homo sapiens; identical to SEQ ID NO: 2

SEQ ID NO: 5 - DNA, 636 bp, Homo sapiens
atggcggcccggcgcggggctctcatagtgctggagggcgtggaccgcgccgggaagagcacgcagagccgcaagctggtggaagcgctgtcgcgcgggccaccgcccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagactcgaagctgtccatgaggaactccgcgtgctctctgaggacgccatccgcactgccacagagaagccgctgggggagctatggaagtga

SEQ ID NO: 6 - protein, 211 aa, Homo sapiens
MAARRGALIVLEGVDRAGKSTQSRKLVEALSRGPPPELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKRLEAVHEELRVLSEDAIRTATEKPLGELWK

SEQ ID NO: 7 - DNA, 639 bp, Homo sapiens
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaaggac

SEQ ID NO: 8 - protein, 213 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWKD

SEQ ID NO: 9 - DNA, 639 bp, Mus musculus
atggcgtcgcgtcggggagcgctcatcgtgctggagggtgtggaccgtgctggcaagaccacgcagggcctcaagctggtgaccgcgctgtgcgcctcgggccacagagcggagctgctgcgtttccccgaaagatcaacggaaatcggcaagcttctgaattcctacttggaaaagaaaacggaactagaggatcactccgtgcacctgctcttctctgcaaaccgctgggaacaagtaccattaattaaggcgaagttgaaccagggtgtgacccttgttttggacagatacgccttttctggggttgccttcactggtgccaaagagaatttttccctggattggtgtaaacaaccggacgtgggccttcccaaacctgacctgatcctgttccttcagttacaattgctggacgctgctgcacggggagagtttggccttgagcgatatgagaccgggactttccaaaagcaggttctgttgtgtttccagcagctcatggaagagaaaaacctcaactggaaggtggttgatgcttccaaaagcattgaggaagtccataaagaaatccgtgcacactctgaggacgccatccgaaacgctgcacagaggccactgggggagctatggaaataa

SEQ ID NO: 10 - protein, 212 aa, Mus musculus
MASRRGALIVLEGVDRAGKTTQGLKLVTALCASGHRAELLRFPERSTEIGKLLNSYLEKKTELEDHSVHLLFSANRWEQVPLIKAKLNQGVTLVLDRYAFSGVAFTGAKENFSLDWCKQPDVGLPKPDLILFLQLQLLDAAARGEFGLERYETGTFQKQVLLCFQQLMEEKNLNWKVVDASKSIEEVHKEIRAHSEDAIRNAAQRPLGELWK

SEQ ID NO: 11 - protein, 212 aa, Homo sapiens
  MISC_FEATURE: F105Y Mutant
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAYTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 12 - protein, 214 aa, Artificial Sequence
  Synthetic construct
  MISC_FEATURE: R16GLL Mutant
MAARRGALIVLEGVDGAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLTPEVGLKRARARGQLDRYENGAFQERALRCFHQLMKDTTLNWKMVDASKSIEAVHEDIRVLSEDAIATATEKPLGELWK

SEQ ID NO: 13 - DNA, 6811 bp, Artificial Sequence
  Synthetic construct
tggaagggctaattcactcccaacgaagacaagatatccttgatctgtggatctaccacacacaaggctacttccctgattggcagaactacacaccaggaccagggatcagatatccactgacctttggatggtgctacaagctagtaccagttgagccagataaggtagaagaggccaacaaaggagagaacaccagcttgttacaccctgtgagcctgcatggaatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcttttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaggaattctgcagtcgagcggagcgcgcgtaatacgactcactatagggcgccatgggtaccgggccccccctcgatcgaacaacaacaacaataacacatggttccgcgtggctctcatatggcggcccggcgcggggctctcatagtgctggagggcgtggacggcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctgcagttaactccggaagttggcttaaaacgcgcacgtgctcgcggcgagcttgaccgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtgaggatcagtcgacggtatcgattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgatatcgaattcctgcagcccgggggatccgccccctctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttaaggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 14 - DNA, 6805 bp, Artificial Sequence
  Synthetic construct
tggaagggctaattcactcccaacgaagacaagatatccttgatctgtggatctaccacacacaaggctacttccctgattggcagaactacacaccaggaccagggatcagatatccactgacctttggatggtgctacaagctagtaccagttgagccagataaggtagaagaggccaacaaaggagagaacaccagcttgttacaccctgtgagcctgcatggaatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcttttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaggaattctgcagtcgagcggagcgcgcgtaatacgactcactatagggcgccatgggtaccgggccccccctcgatcgaacaacaacaacaataacacatggttccgcgtggctctcatatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtgaggatcagtcgacggtatcgattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgatatcgaattcctgcagcccgggggatccgccccctctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttaaggcgcgccccgggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 15 - DNA, 639 bp, Homo sapiens
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 16 - protein, 212 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIATATEKPLGELWK

SEQ ID NO: 17 - protein, 15 aa, Escherichia coli
TPEVGLKRARARGEL

SEQ ID NO: 18 - DNA, 118 bp, HIV 1
  misc_feature: cPPT
ttttaaaagaaaaggggggattggggggtacagtgcaggggaaagaatagtagacataatagcaacagacatacaaactaaagaattacaaaaacaaattacaaaaattcaaaatttt

SEQ ID NO: 19 - DNA, 592 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 20 - protein, 13 aa, Homo sapiens
QLADAAKRGAFGH

SEQ ID NO: 21 - DNA, 639 bp, Homo sapiens
  misc_feature: F105Y mutant
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 22 - DNA, 645 bp, Artificial Sequence
  Synthetic construct
  misc_feature: R16GLL mutant
atggcggcccggcgcggggctctcatagtgctggagggcgtggacggcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctgcagttaactccggaagttggcttaaaacgcgcacgtgctcgcggcgagcttgaccgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 23 - DNA, 28 bp, Homo sapiens
atgccacctcctcgcctcctcttcttcc

SEQ ID NO: 24 - DNA, 22 bp, Homo sapiens
tcacctggtgctccaggtgccc

SEQ ID NO: 25 - DNA, 23 bp, Homo sapiens
ccgccaccgcggtggagctccag

SEQ ID NO: 26 - DNA, 26 bp, Homo sapiens
ttaaagatgaagaatgcccacaaggg

SEQ ID NO: 27 - DNA, 1966 bp, Homo sapiens
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaaaaaaaaaaaaaaaaaa

SEQ ID NO: 28 - protein, 556 aa, Homo sapiens [sequence omitted]

SEQ ID NO: 29 - protein, 313 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 30 - DNA, 939 bp, Homo sapiens
  exon (1)..(939)
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctt

SEQ ID NO: 31 - protein, 313 aa, Homo sapiens
MPPPRLLFFLLFLTPMEVRPEEPLVVKVEEGDNAVLQCLKGTSDGPTQQLTWSRESPLKPFLKLSLGLPGLGIHMRPLASWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 32-33 - skipped (2 entries)

SEQ ID NO: 34 - DNA, 639 bp, Homo sapiens; identical to SEQ ID NO: 21

SEQ ID NO: 35 - DNA, 1878 bp, Homo sapiens
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctt

SEQ ID NO: 36 - protein, 212 aa, Homo sapiens
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAYTGAKENFSLDWCKQPDVGLPKPDLVLFLQLQLADAAKRGAFGHERYENGAFQERALRCFHQLMKDTTLNWKMVDASKSIEAVHEDIRVLSEDAIATATEKPLGELWK

SEQ ID NO: 37 - protein, 313 aa, Homo sapiens; identical to SEQ ID NO: 31

SEQ ID NO: 38 - protein, 531 aa, Artificial Sequence [sequence omitted]

SEQ ID NO: 39 - DNA, 1596 bp, Artificial Sequence
  Synthetic construct
  misc_feature (940)..(957): Linker
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttgccggcggggctgcagggatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga